SEQUENCE LISTING (18 sequences)

SEQ ID NO: 1 - DNA, 22 bp, Artificial
  Synthetic
tggaggatttaacccaggagag

SEQ ID NO: 2 - DNA, 22 bp, Artificial
  Synthetic
tatcatggtcactgggtaggtg

SEQ ID NO: 3 - DNA, 22 bp, Artificial
  Synthetic
ctctcctgggttaaatcctcca

SEQ ID NO: 4 - DNA, 22 bp, Artificial
  Synthetic
cacctacccagtgaccatgata

SEQ ID NO: 5 - DNA, 28 bp, Artificial
  Synthetic
attatcagcacgacagacgcctgaggag

SEQ ID NO: 6 - DNA, 14 bp, Artificial
  Synthetic
gaggtaaccgacca

SEQ ID NO: 7 - DNA, 15 bp, Artificial
  Synthetic
ctccattggctcctn

SEQ ID NO: 8 - DNA, 42 bp, Artificial
  Synthetic
ctcctcaggcgtctgtcgtgctcataatntggtcggtacctc

SEQ ID NO: 9 - DNA, 35 bp, Artificial
  Synthetic
tcggcagaggtggttgtgttgcgatagcccagtat

SEQ ID NO: 10 - DNA, 21 bp, Artificial
  Synthetic
tatcgcaacacaaccacctct

SEQ ID NO: 11 - DNA, 35 bp, Artificial; identical to SEQ ID NO: 9
  Synthetic

SEQ ID NO: 12 - DNA, 21 bp, Artificial
  Synthetic
tatcgcaacacaaccacctct

SEQ ID NO: 13 - DNA, 50 bp, Artificial
  Synthetic
atgctaccgttcaggactggacccgttattacgagatgtccgtcgtcaag

SEQ ID NO: 14 - DNA, 25 bp, Artificial
  Synthetic
ctggacccgttattacgagatgtcc

SEQ ID NO: 15 - DNA, 28 bp, Artificial
  Synthetic
ctcctcaggcgtctgtcgtgctcataat

SEQ ID NO: 16 - DNA, 16 bp, Artificial
  Synthetic
tggtcggttacctcaa

SEQ ID NO: 17 - DNA, 25 bp, Artificial
  Synthetic
tcctgaacggtagcatcttgacgac

SEQ ID NO: 18 - DNA, 18 bp, Artificial
  Synthetic
ctggacccgttattacga

(19) United States
(12) Patent Application Publication
(10) Pub. No.: US 2013/0296172 A1
(43) Pub. Date: Nov. 7, 2013

(54) Methods and Compositions for Analysis of Nucleic Acids

(71) Applicant: AFFYMETRIX, INC., Santa Clara, CA (US)

(72) Inventors: Glenn K. Fu, Dublin, CA (US); Robert G. Kuimelis, Palo Alto, CA (US); Ronald J. Sapolsky, Palo Alto, CA (US)

(21) Appl. No.: 13/793,461

(22) Filed: Mar. 11, 2013

Related U.S. Application Data
(63) Continuation of application No. 13/192,451, filed on Jul. 27, 2011, now abandoned.
(60) Provisional application No. 61/368,236, filed on Jul. 27, 2010.

(51) Int. Cl. C12Q 1/68

(52) U.S. Cl. CPC C12Q 1/6874
USPC 506/2

(57) ABSTRACT

Compositions and methods for analysis of nucleic acids are disclosed. Targets are hybridized to arrays having features that include pairs of co-localized probes within features. The probe pairs may include a first probe type that is oriented so that the 5′ end is free and the 3′ end is attached to the support and a second probe type that is oriented so that the 3′ end is free for extension and the 5′ end is attached to the support. The probes of a feature are complementary to different regions of the same target sequence so they can simultaneously hybridize to a single target with a gap or nick between. The gap may be filled by extension and ligation or ligation.

### RELATED APPLICATIONS

This application claims priority to U.S. Provisional application No. 61/368,236 filed Jul. 27, 2010, the entire disclosure of which is incorporated herein by reference in its entirety.

### FIELD OF THE INVENTION

The present invention relates to the field of molecular biology, and more specifically to methods for nucleic acid amplification and analysis.

### BACKGROUND OF THE INVENTION

With the advent of numerous increasingly affordable DNA sequencing technologies, more and more individual genomes have been sequenced. This explosion of sequence information has led to the discovery of sequence variations from person to person. Most notably, the discovery and characterization of some of these variants, such as Single Nucleotide Polymorphisms, or SNPs, greatly furthers our understanding of phenotype differences from person to person, and the underlying risks and causative mechanisms associated with many diseases. More affordable sequencing technologies have uncovered many differences but there is room for improvement, for example, with respect to accuracy. In most cases, deep sequencing using heavy oversampling is considered to be necessary to improve accuracy of calls. Deep sequencing is an expensive and time consuming solution to tease out the false negatives and positives. More affordable, high-throughput, high-accuracy methods to confirm sequencing calls that were initially discovered in large sequencing efforts would be beneficial.

### SUMMARY OF THE INVENTION

In one aspect methods are disclosed for using solid supports having features that have a first species of 5′ up probe and a second species of 3′ up probe located in the same region so that both probes can hybridize to the same target sequence simultaneously. The hybridized probes on the target are oriented to that the 3′ up probe can be extended on the target in the direction of the hybridized 5′ up probe. In some aspects the gap between the 3′ up probe and the 5′ up probe on the target is filled using a DNA polymerase and the extended 3′ up probe can be joined to the end of the 5′ up probe, eliminating the free ends of the probes.

In one aspect the 5′ up probes and the 3′ up probes are connected at their opposite ends (the 3′ end of the 5′ up probe and the 5′ end of the 3′ end probe) through a common sequence that may be attached to a solid support.

In another aspect, the 5′ up probes and the 3′ up probes are separately connected to the support. The 5′ up probes may have a terminal phosphate and the 3′ up probes may have a terminal hydroxyl group.

In some aspects the 5′ up probe may have a primer binding sequence 3′ of a target specific sequence.

In some aspects the 3′ up probe has one or more cleavable linking groups 5′ of a target specific region. The cleavable linking groups may be used to cleave the 3′ up probe from covalent attachment to the array via the 5′ linking groups.

The features having 5′ up and 3′ up target specific probes can be hybridized to a complementary target so that both are hybridized, the 3′ up probe may be extended by one or more bases that may be labeled and then the ends of the probe can be ligated together to form a single joined probe on the array that has no free ends. The array can be subjected to exonuclease cleavage to remove unligated probes. The 3′ up probes can be cleaved from the array so that only those 3′ up probes that have been ligated to the 5′ up probes will be covalently attached to the solid support. Detection of the ligation event can be detected, for example, by hybridization of a labeled probe that is complementary to a common sequence on the 3′ up probe or by detection of the incorporated label.

In some aspects the 3′ up probe has a target complementary region that is shorter than the target complementary region of the 5′ up probe. In another aspect, the 5′ up probe has a target complementary region that is shorter than the target complementary region of the 3′ up probe. This provides for control of which of the probes binds to the target with greater stability. The lengths may also be similar or identical so that the stability of hybridization is similar or identical.

In some aspects the product resulting from the joining of the ends of the 5′ up probe and the 3′ up probe is analyzed, for example, by sequencing using primer extension and subsequent rounds of single base extension followed by removal of the primer and primer resetting after each step.

Arrays having features that include mixtures of 3′ up and 5′ up probes are disclosed as well as arrays having tethered precircle probes. Kits and reagents for performing the disclosed methods are also contemplated. Kits may include for example, arrays and reagents.

### BRIEF DESCRIPTION OF THE FIGURES

FIG. 1 shows a structure having synthesis points at the 5′ and 3′ end of a detection oligonucleotide for the synthesis of target specific pre circle probes on a solid support.

FIG. 2 shows a schematic of a detection pre circle probe on a solid support.

FIG. 3A shows a method for gap filling and ligation to close a pre circle probe on a solid support.

FIG. 3B shows a gap fill and ligate method performed in parallel with each of the four different nucleotides in a single reaction. A closed circle is formed in one of the four reactions and the other three are unligated and the unligated probes are digested. The ligated probe is detected by hybridization with a labeled detection oligonucleotide.

FIG. 4A shows a schematic of an array of features with a single feature blown up to show the mixture of two probe species in a single feature.

FIG. 4B shows five different possible arrangements for pairs of co-located probes.

FIG. 5A shows a schematic of a two probe, extension, ligation method for genotyping a variation in a target.

FIG. 5B shows a schematic of a sequencing method utilizing co-located pairs of probes.

FIG. 6A shows a schematic of another embodiment for capture of selected targets and extension of the 3′ up probe to make a copy of the target.

FIG. 6B shows sequencing of the extension product from FIG. 6A.

FIG. 7 shows scan images of the hybridization pattern of each of two different oligonucleotides to two copies of the same array.

FIG. 8 shows a schematic of an experiment demonstrating hybridization of probe 2 to a target and extension of that probe in the presence of probe 1 within the same feature. Scan images of fluorescent hybridization are shown on the right.

FIG. 9 shows hybridization of a target to probe 1 and extension of probe 1 in the presence of probe 2 within the same feature. Scan images of fluorescent hybridization are shown on the right.

FIG. 10 shows a schematic on the right and an image of a scan on the left demonstrating bridging of the probes followed by ligation of a labeled reporter to the end of probe 2.

FIG. 11 shows a schematic of a feature that combined an RCA probe for amplification of a target with a sequencing primer.

FIG. 12 shows a feature with an RCA primer for amplification of a target combined with a sequencing primer that is cleavable from the support so that it can be released into solution.

FIG. 13A shows schematics for methods for co-located probes to be used for allele specific analysis for genotyping SNPs and copy number analysis.

FIG. 13B shows a schematic for sequencing or genotyping using pairs of co-located probes without amplification.

FIG. 14A shows a method for cooperative hybridization using co-located probes in different possible orientations on the support.

FIG. 14B is similar to FIG. 14A but there are gaps between the probes when hybridized to the targets.

FIG. 15 shows extension of a 3′ up probe in the presence of klenow and biotin-dUTP, with scans on the bottom and a schematic of the experimental set up above.

FIG. 16 shows the results of a ligation test on a 5′ phosphate probe.

FIG. 17 shows images of scans showing hybridization of labeled probes that are complementary to either the 5′ up probe or the 3′ up probe after cleavage of the diol linkage of the 3′ up probe.

FIG. 18 shows results of whole genome target hybridized to an array of test markers with features having 5′ up and 3′ up probes.

FIG. 19 shows comparison of the signal for probes in their predicted channels compared to the total.

### DETAILED DESCRIPTION

Although the invention is described in conjunction with the exemplary embodiments, the invention is not limited to these embodiments. On the contrary, the invention encompasses alternatives, modifications and equivalents, which may be included within the spirit and scope of the invention. The invention has many embodiments and relies on many patents, applications and other references for details known to those of the art. Therefore, when a patent, application, or other reference is cited or repeated below, the entire disclosure of the document cited is incorporated by reference in its entirety for all purposes as well as for the proposition that is recited. All documents, i.e., publications and patent applications, cited in this disclosure, including the foregoing, are incorporated herein by reference in their entireties for all purposes to the same extent as if each of the individual documents were specifically and individually indicated to be so incorporated herein by reference in its entirety.

As used in this application, the singular form “a,” “an,” and “the” include plural references unless the context clearly dictates otherwise. For example, the term “an agent” includes a plurality of agents, including mixtures thereof.

Throughout this disclosure, various aspects can be presented in a range format. When a description is provided in range format, this is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

The disclosed methods, kits and compositions may employ arrays of probes on solid substrates in some embodiments. Methods and techniques applicable to polymer (including nucleic acid and protein) array synthesis have been described in, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, and in WO 99/36760 and WO 01/58593, which are all incorporated herein by reference in their entirety for all purposes. Patents that describe synthesis techniques include U.S. Pat. Nos. 5,412,087, 6,147,205, 6,262,216, 6,310,189, 5,889,165, and 5,959,098. Nucleic acid probe arrays are described in many of the above patents, but the same techniques may be applied to polypeptide probe arrays.

Nucleic acid arrays that are useful include, but are not limited to, those that are commercially available from Aflymetrix (Santa Clara, Calif.) under the brand name GENECHIP® array. Example arrays are shown on the website at the Affymetrix web site.

Probe arrays have many uses including, but are not limited to, gene expression monitoring, profiling, library screening, genotyping and diagnostics. Methods of gene expression monitoring and profiling are described in U.S. Pat. Nos. 5,800,992, 6,013,449, 6,020,135, 6,033,860, 6,040,138, 6,177,248 and 6,309,822. Genotyping methods, and uses thereof, are disclosed in U.S. patent application Ser. No. 10/442,021 (abandoned) and U.S. Pat. Nos. 5,856,092, 6,300,063, 5,858,659, 6,284,460, 6,361,947, 6,368,799, 6,333,179, and 6,872,529. Other uses are described in U.S. Pat. Nos. 5,871,928, 5,902,723, 6,045,996, 5,541,061, and 6,197,506.

Feature refers to a localized area on a solid support that is, or was, intended to be used for formation of a selected molecule and is otherwise referred to herein in the alternative as a selected or predefined region. The predefined region may have any convenient shape, e.g., circular, rectangular, elliptical, wedge-shaped, etc. For the sake of brevity herein, “features” are sometimes referred to simply as “regions” or “known locations.” In some embodiments, a feature, and therefore the area upon which each distinct compound or group of compounds is synthesized, can be as small as or smaller than 1 micron square as shown in the patents cited above, but is often about 5 microns by 5 microns. Within these regions, the molecule synthesized therein is preferably synthesized in a substantially pure form.

“Solid support”, “support”, and “substrate” refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See the above patents for a broader list of supports.

A “protective group” is a moiety which is bound to a molecule and which may be spatially removed upon selective exposure to an activator such as electromagnetic radiation. Several examples of protective groups are known in the literature and will become evident upon further reading of the present disclosure. Other examples of activators include ion beams, electric fields, magnetic fields, electron beams, x-ray, and the like.

Activating group refers to those groups which, when attached to a particular functional group or reactive site, render that site more reactive toward covalent bond formation with a second functional group or reactive site. For example, the group of activating groups which can be used in the place of a hydroxyl group include —O(CO)Cl; —OCH₂Cl; —O(CO)OAr, where Ar is an aromatic group, preferably, a p-nitrophenyl group; —O(CO)(ONHS); and the like. The group of activating groups which are useful for a carboxylic acid include simple ester groups and anhydrides. The ester groups include alkyl, aryl and alkenyl esters and in particular such groups as 4-nitrophenyl, N-hydroxylsuccinimide and pentafluorophenol. Other activating groups are known to those of skill in the art.

Samples can be processed by various methods before analysis. Prior to, or concurrent with, analysis a nucleic acid sample may be amplified by a variety of mechanisms, some of which may employ PCR. (See for example, PCR Technology: Principles and Applications for DNA Amplification, Ed. H. A. Erlich, Freeman Press, NY, N.Y., 1992; PCR Protocols: A Guide to Methods and Applications, Eds. Innis, et al., Academic Press, San Diego, Calif., 1990; Mattila et al., Nucleic Acids Res., 19:4967, 1991; Eckert et al., PCR Methods and Applications, 1:17, 1991; PCR, Eds. McPherson et al., IRL Press, Oxford, 1991; and U.S. Pat. Nos. 4,683,202, 4,683,195, 4,800,159 4,965,188, and 5,333,675, each of which is incorporated herein by reference in their entireties for all purposes. The sample may also be amplified on the probe array. (See, for example, U.S. Pat. No. 6,300,070 and U.S. patent application Ser. No. 09/513,300 (abandoned), all of which are incorporated herein by reference).

Other suitable amplification methods include the ligase chain reaction (LCR) (see, for example, Wu and Wallace, Genomics, 4:560 (1989), Landegren et al., Science, 241:1077 (1988) and Barringer et al., Gene, 89:117 (1990)), transcription amplification (Kwoh et al., Proc. Natl. Acad. Sci. USA, 86:1173 (1989) and WO 88/10315), self-sustained sequence replication (Guatelli et al., Proc. Nat. Acad. Sci. USA, 87:1874 (1990) and WO 90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909 and 5,861,245) rolling circle amplification (RCA) (for example, Fire and Xu, PNAS 92:4641 (1995) and Liu et al., J. Am. Chem. Soc. 118:1587 (1996)) and nucleic acid based sequence amplification (NABSA). (See also, U.S. Pat. Nos. 5,409,818, 5,554,517, and 6,063,603, each of which is incorporated herein by reference). Other amplification methods that may be used are described in, for instance, U.S. Pat. Nos. 6,582,938, 5,242,794, 5,494,810, and 4,988,617, each of which is incorporated herein by reference.

Other amplification methods that may be used are described in, U.S. Pat. Nos. 5,242,794, 5,494,810, 4,988,617 and in U.S. Ser. No. 09/854,317. Other amplification methods are also disclosed in Dahl et al., Nuc. Acids Res. 33(8):e71 (2005) and circle to circle amplification (C2CA) Dahl et al., PNAS 101:4548 (2004). Locus specific amplification and representative genome amplification methods may also be used. US Patent Pub. No. 20090117573 discloses methods for multiplex amplification of targets using arrayed probes.

Additional methods of sample preparation and techniques for reducing the complexity of a nucleic sample are described in Dong et al., Genome Research, 11:1418 (2001), U.S. Pat. Nos. 6,361,947, 6,391,592, 6,632,611, 6,872,529 and 6,958,225, and in U.S. patent application Ser. No. 09/916,135 (abandoned).

Hybridization assay procedures and conditions vary depending on the application and are selected in accordance with known general binding methods, including those referred to in Maniatis et al., Molecular Cloning: A Laboratory Manual, 2^{nd} Ed., Cold Spring Harbor, N.Y., (1989); Berger and Kimmel, Methods in Enzymology, Guide to Molecular Cloning Techniques, Vol. 152, Academic Press, Inc., San Diego, Calif. (1987); Young and Davism, Proc. Nat'l. Acad. Sci., 80:1194 (1983). Methods and apparatus for performing repeated and controlled hybridization reactions have been described in, for example, U.S. Pat. Nos. 5,871,928, 5,874,219, 6,045,996, 6,386,749, and 6,391,623 each of which are incorporated herein by reference.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5X SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na+], 20 mM EDTA, 0.01% Tween-20 and a temperature of 30-50° C., or at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GENECHIP® Mapping Assay Manual, 2004.

Hybridization signals can be detected by conventional methods, such as described by, e.g., U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,225,625, U.S. patent application Ser. No. 10/389,194 (U.S. Patent Application Publication No. 2004/0012676, allowed on Nov. 9, 2009) and PCT Application PCT/US99/06097 (published as WO 99/47964), each of which is hereby incorporated by reference in its entirety for all purposes).

The practice of the methods may also employ conventional biology methods, software and systems. Computer software products of the invention typically include, for instance, computer readable medium having computer-executable instructions for performing the logic steps of the method of the invention. Suitable computer readable medium include, for example a floppy disk, CD-ROM/DVD/DVD-ROM, hard-disk drive, flash memory, ROM/RAM, and magnetic tapes. The computer executable instructions may be written in a suitable computer language or combination of several computer languages. Basic computational biology methods which may be employed in the methods are described in, for example, Setubal and Meidanis et al., Introduction to Computational Biology Methods, PWS Publishing Company, Boston, (1997); Salzberg, Searles, Kasif, (Ed.), Computational Methods in Molecular Biology, Elsevier, Amsterdam, (1998); Rashidi and Buehler, Bioinformatics Basics: Application in Biological Science and Medicine, CRC Press, London, (2000); and Ouelette and Bzevanis Bioinformatics: A Practical Guide for Analysis of Gene and Proteins, Wiley & Sons, Inc., 2^{nd} ed., (2001). (See also, U.S. Pat. No. 6,420,108).

The invention may also make use of various computer program products and software for a variety of purposes, such as probe design, management of data, analysis, and instrument operation. (See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170).

Genetic information obtained can be transferred over networks such as the internet, as disclosed in, for instance, (U.S. Patent Application Publication No. 20030097222), U.S. Patent Application Publication No. 20020183936, abandoned), U.S. Patent Application Publication No. 20030100995, U.S. Patent Application Publication No. 20030120432, Ser. No. 10/328,818 U.S. Patent Application Publication No. 20040002818, U.S. Patent Application Publication No. 20040126840, abandoned), Ser. No. 10/423,403 (U.S. Patent Application Publication No. 20040049354.

Methods for multiplex amplification and analysis of nucleic acids have been disclosed, for example in U.S. Pat. Nos. 6,858,412 and 7,700,323. Related methods are also disclosed in U.S. Pat. Nos. 6,558,928, 6,235,472, 6,221,603, 5,866,337, and 4,988,617. Applications of MIP technology have been described in, for example, Daly et al. Clin Chem 2007, 53(7): 1222-1230, Dumaual, et al. Pharmacogenomics 2007, 8(3):293-305, Ireland et al. Hum Genet 2006, 119:75-83, Moorhead et al. Eur. J. Hum Genet 2006, 14:207-215, Hardenbol, et al., Genome Rev. 2005, 15:269-275 and Hardenbol, et al. Nat. Biotech. 2003, 21:673-678 and Wang et al. NAR 33:e183.

Many of the methods and systems disclosed herein utilize enzyme activities. A variety of enzymes are well known, have been characterized and many are commercially available from one or more supplier. For a review of enzyme activities commonly used in molecular biology see, for example, Rittie and Perbal, J. Cell Commun. Signal. (2008) 2:25-45, incorporated herein by reference in its entirety. Exemplary enzymes include DNA dependent DNA polymerases (such as those shown in Table 1 of Rittie and Perbal), RNA dependent DNA polymerase (see Table 2 of Rittie and Perbal), RNA polymerases, ligases (see Table 3 of Rittie and Perbal), enzymes for phosphate transfer and removal (see Table 4 of Rittie and Perbal), nucleases (see Table 5 of Rittie and Perbal), and methylases.

The term “Strand Displacement Amplification” (SDA) is an isothermal in vitro method for amplification of nucleic acid. In general, SDA methods initiate synthesis of a copy of a nucleic acid at a free 3′ OH that may be provided, for example, by a primer that is hybridized to the template. The DNA polymerase extends from the free 3′ OH and in so doing, displaces the strand that is hybridized to the template leaving a newly synthesized strand in its place. Subsequent rounds of amplification can be primed by a new primer that hybridizes 5′ of the original primer or by introduction of a nick in the original primer. Repeated nicking and extension with continuous displacement of new DNA strands results in exponential amplification of the original template. Methods of SDA have been previously disclosed, including use of nicking by a restriction enzyme where the template strand is resistant to cleavage as a result of hemimethylation. Another method of performing SDA involves the use of “nicking” restriction enzymes that are modified to cleave only one strand at the enzymes recognition site. A number of nicking restriction enzymes are commercially available from New England Biolabs and other commercial vendors.

Polymerases useful for SDA generally will initiate 5′ to 3′ polymerization at a nick site, will have strand displacing activity, and preferably will lack substantial 5′ to 3′ exonuclease activity. Enzymes that may be used include, for example, the Klenow fragment of DNA polymerase I, Bst polymerase large fragment, Phi29, and others. DNA Polymerase I Large (Klenow) Fragment consists of a single polypeptide chain (68 kDa) that lacks the 5′ to 3′ exonuclease activity of intact E. coli DNA polymerase I. However, DNA Polymerase I Large (Klenow) Fragment retains its 5′ to 3′ polymerase, 3′ to 5′ exonuclease and strand displacement activities. The Klenow fragment has been used for SDA. For methods of using Klenow for SDA see, for example, U.S. Pat. Nos. 6,379,888; 6,054,279; 5,919,630; 5,856,145; 5,846,726; 5,800,989; 5,766,852; 5,744,311; 5,736,365; 5,712,124; 5,702,926; 5,648,211; 5,641,633; 5,624,825; 5,593,867; 5,561,044; 5,550,025; 5,547,861; 5,536,649; 5,470,723; 5,455,166; 5,422,252; 5,270,184, the disclosures of which are incorporated herein by reference. There are many thermostable polymerases and polymerase mixtures that are commercially available and may be used in combination with the disclosed methods.

Phi29 is a DNA polymerase from Bacillus subtilis that is capable of extending a primer over a very long range, for example, more than 10 Kb and up to about 70 Kb. This enzyme catalyzes a highly processive DNA synthesis coupled to strand displacement and possesses an inherent 3′ to 5′ exonuclease activity, acting on both double and single stranded DNA. Variants of phi29 enzymes may be used, for example, an exonuclease minus variant may be used. Phi29 DNA Polymerase optimal temperature range is between about 30° C. to 37° C., but the enzyme will also function at higher temperatures and may be inactivated by incubation at about 65° C. for about 10 minutes. Phi29 DNA polymerase and Tma Endonuclease V (available from Fermentas Life Sciences) are active under compatible buffer conditions. Phi29 is 90% active in NEB buffer 4 (20 mM Tris-acetate, 50 mM potassium acetate, 10 mM magnesium acetate and 1 mM DTT, pH 7.9 at 25° C.) and is also active in NEBuffer 1 (10 mM Bis-Tris-Propane-HCl, 10 mM magnesium chloride and 1 mM DTT, pH 7.0 at 25° C.). NEBuffer 2 (50 mM sodium chloride, 10 mM Tris-HCl, 10 mM magnesium chloride and 1 mM DTT, pH 7.9 at 25° C.), NEB Buffer 3(100 mM NaCl, 50 mM Tris HCl, 10 mM magnesium chloride and 1 mM DTT, pH 7.9 at 25° C.). For additional information on phi29, see U.S. Pat. Nos. 5,100,050, 5,198,543 and 5,576,204.

Bst DNA polymerase originates from Bacillus stearothermophilus and has a 5′ to 3′ polymerase activity, but lacks a 5′ to 3′ exonuclease activity. This polymerase is known to have strand displacing activity. The enzyme is available from, for example, New England Biolabs. Bst is active at high temperatures and the reaction may be incubated optimally at about 65° C. but also retains 30%-45% of its activity at 50° C. Its active range is between 37° C. and 80° C. The enzyme tolerates reaction conditions of 70° C. and below and can be heat inactivated by incubation at 80° C. for 10 minutes. Bst DNA polymerase is active in the NEBuffer 4 (20 mM Tris-acetate, 50 mM potassium acetate, 10 mM magnesium acetate and 1 mM DTT, pH 7.9 at 25° C.) as well as NEBuffer 1 (10 mM Bis-Tris-Propane-HCl, 10 mM magnesium chloride and 1 mM DTT, pH 7.0 at 25° C.), NEBuffer 2(50 mM sodium chloride, 10 mM Tris-HCl, 10 mM magnesium chloride and 1 mM DTT, pH 7.9 at 25° C.), and NEBuffer 3(100 mM NaCl, 50 mM Tris HCl, 10 mM magnesium chloride and 1 mM DTT, pH 7.9 at 25° C.). Bst DNA polymerase could be used in conjunction with E. coli Endonuclease V (available from New England Biolabs). For additional information see Mead, D. A. et al. (1991) BioTechniques, p.p. 76-87, McClary, J. et al. (1991) J. DNA Sequencing and Mapping, p.p. 173-180 and Hugh, G. and Griffin, M. (1994) PCR Technology, p.p. 228-229.

Endonucleases are enzymes that cleave a nucleic acid (DNA or RNA) at internal sites in a nucleotide base sequence. Cleavage may be at a specific recognition sequence, at sites of modification or randomly. Specifically, their biochemical activity is the hydrolysis of the phosphodiester backbone at sites in a DNA sequence. Examples of endonucleases include Endonuclease V (Endo V) also called deoxyinosine 3′ endonuclease, which recognizes DNA containing deoxyinosines (paired or not). Endonuclease V cleaves the second and third phosphodiester bonds 3′ to the mismatch of deoxyinosine with a 95% efficiency for the second bond and a 5% efficiency for the third bond, leaving a nick with 3′ hydroxyl and 5′ phosphate. Endo V, to a lesser, degree, also recognizes DNA containing abasic sites and also DNA containing urea residues, base mismatches, insertion/deletion mismatches, hairpin or unpaired loops, flaps and pseudo-Y structures. See also, Yao et al., J. Biol. Chem., 271(48): 30672 (1996), Yao et al., J. Biol. Chem., 270(48): 28609 (1995), Yao et al., J. Biol. Chem., 269(50): 31390 (1994), and He et al., Mutat. Res., 459(2): 109 (2000). Endo V from E. coli is active at temperatures between about 30 and 50° C. and preferably is incubated at a temperature between about 30° C. to 37° C. Endo V is active in NEBuffer 4 (20 mM Tris-acetate, 50 mM potassium acetate, 10 mM magnesium acetate and 1 mM DTT, pH 7.9 at 25° C.), but is also active in other buffer conditions, for example, 20 mM HEPES-NaOH (pH 7.4), 100 mM KCl, 2 mM MnCl.sub.2 and 0.1 mg/ml BSA. Endo V makes a strand specific nick about 2-3 nucleotides downstream of the 3′ side of inosine base, without removing the inosine base. Endonucleases, including Endo V, may be obtained from manufacturers such as New England Biolabs (NEB) or Fermentas Life Sciences. The enzyme Uracil-DNA Glycosylase (UDG or UNG) catalyzes the hydrolysis of the N-glycosylic bond between the uracil and sugar, leaving an a pyrimidinic site in uracil-containing single or double-stranded DNA. This activity has been used, for example, for site directed mutation (Kunkel, PNAS 82:488-492 (1985) and for elimination of PCR carry-over contamination (Longo, et al., Gene 93:125-128 (1990). Uracil mediated cleavage has also been used for cleaving single stranded circularized probes (Hardenbol et al., Genome Res. 15:269-75 (2005).

In one aspect, methods are disclosed for synthesizing and analyzing molecular inversion probes (MIPS) directly on a solid support. In preferred aspects the synthesis is a photolithographic synthesis as described in, for example, in U.S. Pat. Nos. 5,412,087, 6,147,205, 6,262,216, 6,310,189, 5,889,165, and 5,959,098. The MIP assay is well described in the art, see for example U.S. Pat. No. 6,858,412 and Hardenbol, et al., Genome Res. 2005, 15:269-275, each of which is incorporated herein in its entirety for all purposes, particularly for the purpose of describing the MIP assay.

A panel of oligonucleotide probes may be developed, each with the following properties: the 5′ and 3′ arms can anneal to target domains on either side of a genomic SNP or other region to be analyzed. The probe, also referred to herein as a precircle probe, is added to a target sequence from a sample that contains the target domains to form a hybridization complex. The target domains in the target sequence can be directly adjacent, or can be separated by a gap of one or more nucleotides. The precircle probe comprises first and second targeting domains at its termini that are substantially complementary to the target domains of the target sequence. The precircle probe may also include one or optionally more universal priming sites, separated by a cleavage site, and a barcode sequence. If there is no gap between the target domains of the target sequence, and the 5′ and 3′ nucleotides of the precircle probe are perfectly complementary to the corresponding bases at the junction of the target domains, then the 5′ and 3′ nucleotides of the precircle probe are “abutting” each other and can be ligated together, using a ligase, to form a closed circular probe. The 5′ and 3′ end of a nucleic acid molecule are referred to as “abutting” each other when they are in contact close enough to allow the formation of a covalent bond, in the presence of ligase and adequate conditions.

In some aspects there is a one-base gap between the ends of the probe and the SNP so that the SNP position is initially not hybridize to the probe. In another aspect the gap may be greater than a single base and in other aspects the probe may hybridize to the SNP position and the probe may be allele specific, e.g. a first probe that is complementary to a first allele and a second probe that is complementary to a second allele of the SNP. If there is a single base gap a gap-fill formulation (with polymerase and ligase) can fill in this gap if provided with the correct single dNTP whereas the other three dNTPs will not fill the gap. A ligase activity is used to join the ends of the MIP and results in a closed circle conformation. An exonuclease may be used to destroy all MIPs in which the gap has not been filled and the ends of the MIP ligated to close the circle. Subsequent enzymatic reactions, such as PCR amplification or RCA may be used to isolate the one-of-four MIPs that survive and to detect an accompanying “tag” sequence on the MIP (each in the panel unique to its own SNP) upon a universal tag array (whether mounted in a cartridge or on a peg).

The methods disclosed herein provide an alternative means for performing MIP assays using a solid support. To prepare MIP panels for solutions based MIP a unique oligonucleotide of length approximately 115 to 125 nt is required for each target to be analyzed. The probes each have with two unique homology regions flanking the SNP position, a unique tag sequence, and two common regions complementary to amplification primers. As the number of targets to be analyzed in a single assay increases so does the number of MIPs that need to be synthesized to perform the assay. Methods are disclosed herein for improved synthesis methods for the MIPs. Because the probes are attached to a solid support in known or determinable locations the unique barcode region can be omitted. The universal priming sequences are also not required. As a result the precircle probes can be considerably shorter than the comparable probe for solution based assays. In some aspects more than 100,000 MIPs may be generated and assayed using photolithography to synthesize the probes.

Methods for synthesizing MIPs on a microarray with the intention of shearing them off upon completion to create the probe pool in situ has previously been disclosed. A challenge with this approach has been the efficiency of synthesis of probes of the needed length, greater than 100 bases. Improved synthesis methods and chemistries can be used to minimize non-full length probes and quality control assays may be used to monitor efficiency of full-length or nearly full length synthesis.

Disclosed herein are methods for utilizing MIPs that are still attached to the feature of the array in which the MIP was synthesized. This eliminates the need to cleave the MIP from the array, eliminates the need to include a tag for subsequent identification of the amplification product and eliminates the need to include PCR primers in the MIP. The MIP may as a result be considerably shorter and as a result there will be more full length probes on the array. At each synthesis step some number of probes is lost because they don't get the base added in that step-fewer steps results in less probes left behind.

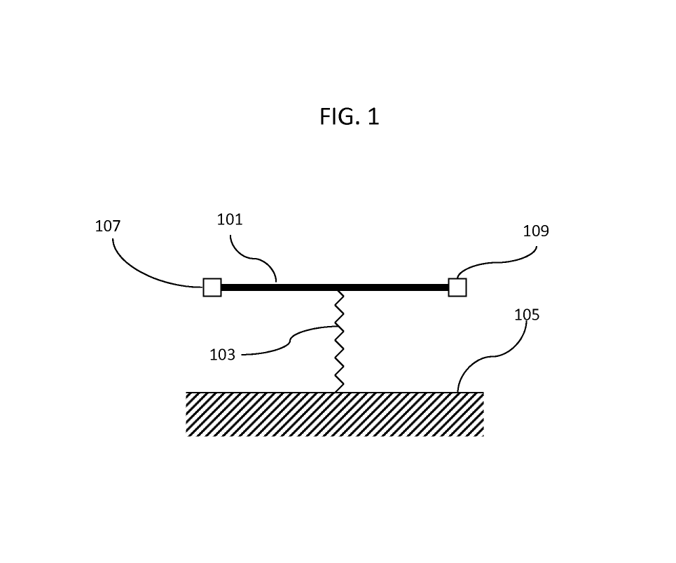

In general one aspect of the methods includes the following steps. First, the surface of the array, whether destined for cartridges or for pegs, is derivatized as follows: a DNA sequence 101 complementary to a common detection oligo (˜15 to 40 bases in length) is tethered at its center to a linker 103 that attaches or is attached to the common oligo and to the array surface 105 over many or all of the features of the array. The 5′ 107 and 3′ ends 109 of this oligo each have a blocking group for use with photolithographic synthesis methods. One for synthesis in the 5′-to-3′ direction and the other for synthesis in the 3′-to-5′ direction. In preferred aspects the entire array has a relatively uniform density (e.g. a lawn) of this template for synthesis so the chemistry used to attach it to the surface need not be photolithographic (see FIG. 1). In some aspects a branched structure, e.g. a branched nucleotide is used to connect the liner 103 to the common oligo 101. This common sequence 101 is complementary to a common detection oligo that can be hybridized to the array at a later step to detect the presence of the common sequence. The detection oligo can be labeled, for example, with a biotin or a hapten.

Photolithography is used in two processes that may be separate or simultaneous: (1) to “grow” from the 5′ end (3′-to-5′ synthesis) the H1 sequence 201 complementary to the genomic DNA flanking a SNP and (2) to “grow” from the 3′ end (5′-to-3′ synthesis) the H2 sequence 203 complementary to the genomic DNA flanking a SNP or other target region to be analyzed. The H1 and H2 regions may each contain a region of about 15 to 30 bases that is preferably perfectly complementary to the target. The H1 and H2 regions may also include linker regions that are not complementary to the target that link the target complementary region to the common sequence. That region is not required and is preferably short, like less than 10 bases.

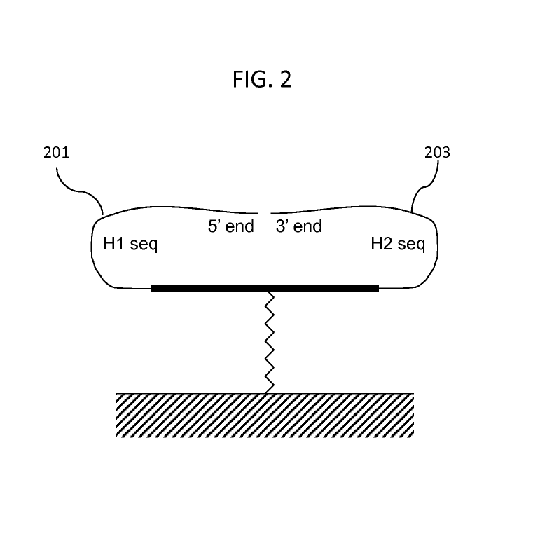

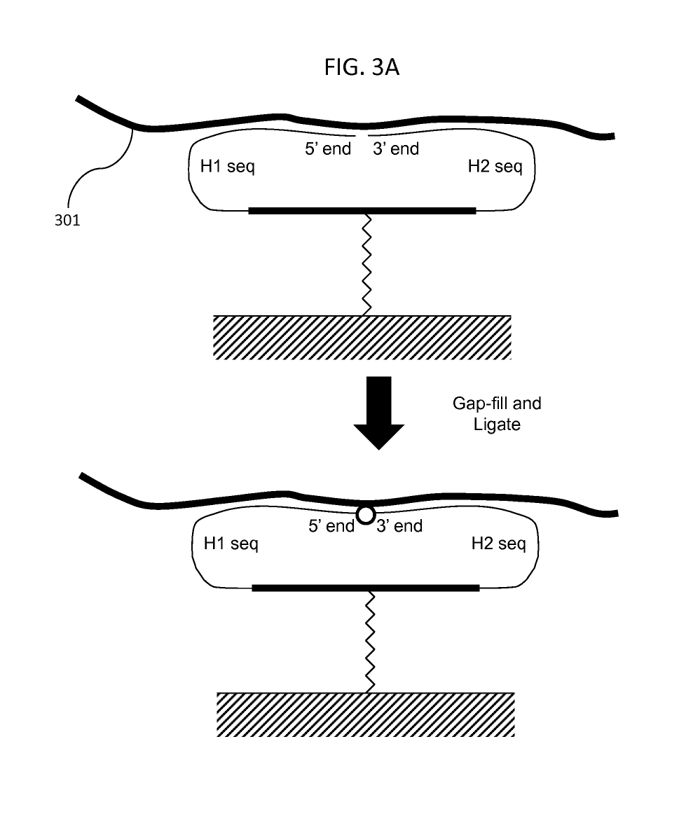

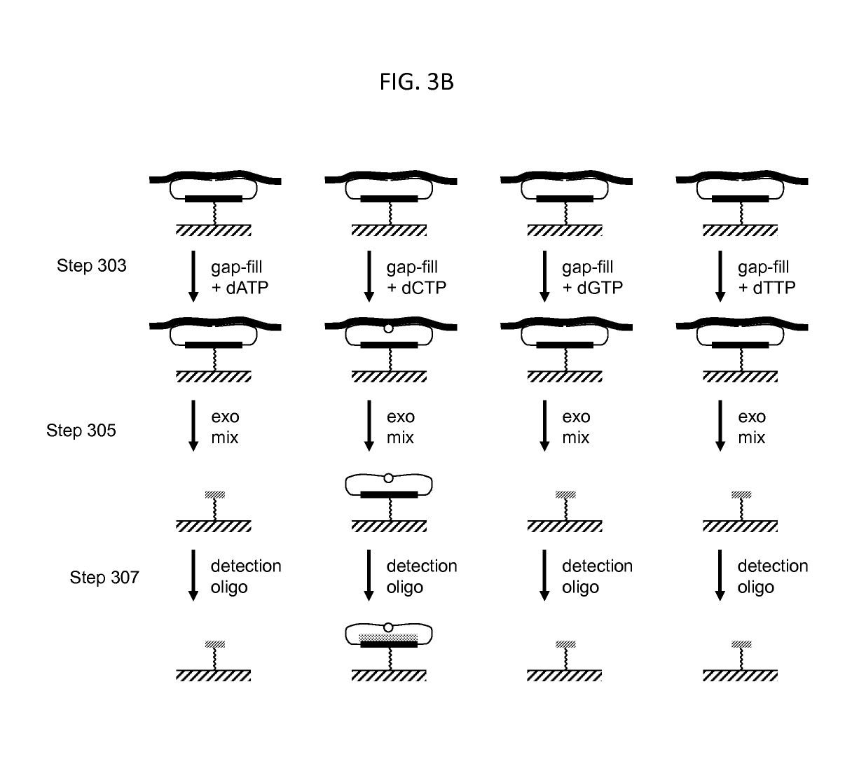

After synthesis each feature of the array now contains hundreds of thousands of oligos, each having the genomic regions flanking a SNP and having a common detection sequence (see FIG. 2). If the oligos are not full-length on both ends, then the resulting gap surrounding the SNP will not be a single nucleotide gap. The MIP assay can then be performed as shown schematically in FIG. 3A. The target 301 is hybridized to both the H1 and H2 simultaneously so that the 5′ and 3′ ends are juxtaposed on the target. If there is a gap it can be filled by one or more bases and the nick sealed by ligation (the small open circle in the figure represents the covalent bond formed between the two ends by the ligation reaction to join H1 and H2 regions and to form a closed circle. The reaction is shown in FIG. 3B as a 4×1 color assay for detection of a G/G SNP. Each column is a different reaction on a different solid support. In step 303 the 5′ end of the tethered MIP (the H1 arm) is kinased, for example with ATP and a polynucleotide-kinase, for example, T4 PNK. Genomic DNA is added along with appropriate buffers for annealing (1× Buffer A+Enzyme A apyrase) incubated 5 minutes at 20° C., denatured for 5 minutes at 95° C., and cooled to 58° C. For four-array/one-color detection schemes, the genomic DNA in anneal mix is hybridized to four arrays at 58° C. overnight with mixing. For N samples, there will be 4N chips. The arrays will be cooled and gap-fill mix will be added to all arrays, then incubated at 58° C. for about 10-15 minutes (more preferably 11 min) with mixing.

The arrays will be cooled and the four arrays in each sample will each receive one dNTP (A, C, G or T). The reaction is then incubated at 58° C. for about 10-15 minutes (more preferably 11 min) with mixing. At this point, the full-length probes in each feature will be circularized by gap-filling the correct nucleotide on one of the four arrays followed by ligation to close the circle. On the other three arrays the probes of that feature will remain linear. If the SNP is biallelic the precircle probe may be closed on two different arrays.

In step 305 the arrays are cooled and exonuclease activity is added to all arrays, then incubated at 37° C. for 15 minutes with mixing. At this point, the circularized probes in each feature will remain intact, resistant to exonuclease; the non-circularized probes (including all non-full-length probes that fail to gap-fill, as well as annealed genomic DNA) will be destroyed. It is important that the action of the exonuclease proceeds a significant distance into the common detection sequence to which the linking tether was attached to the array.

In step 307 the arrays are washed and hybridized with a standard biotin detection oligo. In each quartet of arrays per sample, the detection oligo will hybridize to the one-in-four probes at each feature which received the appropriate gap-fill. Standard staining protocol with SAPE follows. The arrays are scanned. Detection and analysis of SNP genotypes proceeds much in the same way as for 4-array/1-color MIP assays. In the example shown, the SNP is a homozygous G so the detection oligo is detected above background levels only in the reaction where dCTP was added. If the SNP were heterozygous you would expect signal in 2 of the 4 reactions.

This methodology has the following advantages: (1) there is no need to synthesize tens or hundreds of thousands of MIP oligonucleotides separately, followed by single-plex ligation reactions, to create probe panels, thus drastically reducing the cost of probe production; (2) there is no need to account for tag sequences or tag sequence detection in the assay since SNPs are now simply identified by the unique feature position on the MIP on the array; (3) there is no need for amplification steps after the exonuclease reaction, greatly simplifying the MIP assay.

In some aspects there may be optimization required to insure that the detection oligo hybridizes to a complementary sequence tethered to the array at its center. IN some aspects, the tether can be positioned off-center on the common oligo so as not to interfere with the detection sequence.

In many of the embodiments disclosed herein, the features of the array have probes that are synthesized both in the 5′ up direction and the 3′ up direction. In many aspects the synthesis process generates oligo-DNA probes using nucleoside monomers protected with photo-removable groups. Irradiation of the partially built oligomer with near-UV wavelengths deprotects the terminal group and the use of masks allows for control of the sequence of the probe and the size of the features. Different photo-removable protecting groups can be used. See, for example, Afroz et al. Clinical Chem. 50:1936-1939 (2004) and McGall et al. J Am Chem Soc 119:5081-5090 (1997). See also US 20050164258

In some aspects steps are taken to mitigate degradation of the products that might result from incubation at 58° C. overnight for the annealing of the genomic DNA. Improved glues that prevent separation of the array from the cartridge may be used for cartridge arrays. Peg mounted arrays such as those available for use on the GENETITAN instrument system would not require any modification for such treatment.

In some aspects the gap-fill steps may be optimized for function in combination with the array surface. The density of the array bound MIPs is optimized for fill-in and ligation in some aspects. In some aspects the exonuclease mix is optimized to work efficiently on the surface of the array. It will be desirable to determine conditions such that detection of the oligo sequence at the center of the linear MIP probes is efficiently destroyed so that background and noise is sufficiently low.

In some aspects the tethered circles may be amplified, for example, using rolling circle amplification (RCA) methods. Labeled concatemeric DNA amplification products that remain annealed to the features where it is synthesized may be detected.

In another aspect the tethered detection probes are attached to particles that may be encoded, for example, those disclosed in U.S. Pat. Nos. 7,745,092 and 7,745,091. Each MIP may be associated with a particular code associated with the particle. The code may be read in a variety of methods, for example, optically.

Hybridization, Extension, Ligation and Sequencing (HXLS).

In the quest to enable the sequencing of an entire human genome for under $1000, many new technologies are being developed and optimized by various institutions and commercial entities. Next-generation sequencing (NGS) technologies that have been developed include those of Illumina/Solexa, Life Technoloies (ABI), Ion Torrent, Roche 454 and Helicos. For a review of sequencing technologies see, for example, Metzker, M L, Nature Rev. Genet., 11:31-46 (2010), which is incorporated herein by reference in its entirety. While each is unique in the technology, all incorporate a massively parallel approach in order to accomplish sequencing at low cost. In these technologies, short fragments of random DNA are sequenced and then assembled together into a contiguous longer DNA sequence assembly. The disadvantage of these technologies is that each short fragment is essentially a random piece of DNA and in order to completely sequence any given region within the genome test sample, a large sampling redundancy is required. Secondly, there is no capability to avoid the repetitive, non-informative regions of the genome as sampling is random in nature.

Related methods are disclosed in U.S. patent application Ser. Nos. 12/822,179 published as US Pat. Pub. 20100323914, 12/402,486 published as US Pat. Pub. 20090239764 and 12/211,100 published as US Pat Pub. 20090117573, each of which is incorporated herein by reference in its entirety for all purposes.

In order to solve this problem, locus-specific probes can be used to target the regions of interest. One efficient method to generate highly multiplexed arrays of locus-specific probes is through in-situ synthesis, with one example being the photolithographic pocess used to produce Affymetrix GENECHIP arrays. Although the genome regions of interest can hybridize specifically to the arrayed probes and be detectable, the number of molecules (estimated to be in the hundreds or thousands at the maximum) is insufficient to conduct biochemical assays that deduce the sequence composition of hybridized molecules. This described invention is a method to enable solid-phase locus specific amplification of limiting amounts of target molecules hybridized to arrayed probes. The hybridized target molecules are amplified while they remain specifically hybridized to the arrayed probes. Post solid-phase amplification, the amplified DNAs can then be assayed by methods similar to any of those used by the above mentioned technologies. This invention makes possible locus-specific, low redundancy sequencing of genomic regions of interest or whole genomes.

In some aspects, the steps of the method are as follows: First, sample DNA is hybridized to a reverse probe (5′ to 3′ probes) array. Specific DNA that is hybridized is used as template in an extension assay. A DNA polymerase is used to extend the arrayed primer to the end of the hybridized target. The hybridized target is removed via denaturation. The end of the extended primer is attached to an oligonucleotide, for example by ligation with a DNA ligase. The attached oligonucleotide may contain nicking or cleaving restriction enzyme sites, universal sequences for priming, hairpin sequences, or a RNA polymerase promoter sequence such as T7, T3 or Sp6. By exploiting the attached oligonucleotide sequence, the extended probe can be made double-stranded using DNA polymerase. The double stranded DNA may then be used as template for strand-displacement, bridge-amplification, or in vitro transcription amplification reactions. Amplified DNAs (or RNAs) hybridize to adjacent array probes as they get synthesized in the same physical space and the process may in some aspects be repeated in cyclical fashion. The end-result may be solid-phase amplification of locus-specific genomic sequences. Amplified sequences can then be assayed by various biochemical methods such as single base extension or ligation assays using the same arrayed probes used for solid-phase amplification.

Genotyping has become an increasingly valuable tool in our quest to understand the phenotypes that make individuals unique and that result in disease. There are thought to be at least 6 million SNPs in the human genome and current genotyping methods are not able to assay every SNP. Some methods can efficiently assay only about half of the known SNPs. Some markers resolve poorly on give assay platforms. Next generation sequencing methods available currently may not be able to localize regions of interest efficiently and have relatively poor accuracy at low sampling depths. The combination of hybridization plus post capture processing using enzymatic methods may facilitate improvements on these current methods. Array based methods disclosed herein employ target capture and on-array sample prep without amplification.

In another aspect methods are disclosed that incorporate a combination of methods to generate high-accuracy base calls on-demand, for any position in the genome. The methods utilize DNA probes on microarrays to capture the region or locus of interest on a first target specific probe. Next, as not all of the target DNA captured by hybridization is necessarily the exact DNA of interest, a second array probe in the vicinity (about 10 nm distance away) is used to direct a “primer” to only those DNA molecules of interest. At this point, a DNA polymerase is used to extend and fill a gap between the first and second array probes. The gap may be a single nucleotide.

Additionally, a DNA ligase may be used to join only perfect-matching extended nucleotides from the second array probe to the first array probe. Differential labeling of the nucleotides used by the DNA polymerase makes possible identification of the base present at the gap. In the figure each of the nucleotides has a different label (indicated as &, $, # or *). Each label is differentially detectable, for example, each may be detectable at a different wavelength or emit at a different wavelength. In some aspects, the assay has the following steps: hybridization, extension, ligation and sequencing and may be abbreviated as HXLS.

Some of the challenges observed with extension based approaches to genotyping or sequencing include formation of 3′ end self-hairpins or intermolecular dimmers that lead to target independent extension and low specificity and 3′ end truncated probes resulting in incorrect position readout. Problems with ligation based approaches to sequencing and genotyping include excessive target-independent ligation background resulting in high signal in the absence of target and probes on the array forming intra or inter base pairing to result in ligation. Also, insufficient signals due to low concentrations of matching randomers (solution probes), for example, with N8 randomers only 1 in 65,536 solution probes will match the ligation site perfectly. High concentrations of solution probes used in the assay lead to high background, solution probes hybridize to probes or sticking non-specifically. Ligase is permissive to mis-match ligation under the conditions used for the assay. This has been demonstrated with oligonucleotides that are mismatched at the site of ligation discrimination. The 3′ end can form self-hairpins or intermolecular dimmers leading to target independent extension. In some methods the probes may be 3′ end truncated.

In some aspects, chemically cleavable nucleotide analogues with reversible terminators can be used for sequencing. Preferably each base has a different label, for example, a different detectable color of fluorescence. For examples of reversible terminators see, for example, Ju et al. PNAS 103(52):19635-40 (2006) and Litosh et al. Nucleic Acids Res. 39(6):e39 (2011).

Advantages of the HXLS method include, for example, the removal of target independent signals through the elimination of solutions probes. The methods have high specificity of priming from adjacent 3′ OH probes, leading to high sensitivity. The methods have a dramatic reduction in non-specific background because the assay has 0.1 μM dNTPs instead of a 20 μM solution of probes. Self extension from 3′ OH probes is minimized prior to detection. Target captured by 5′ phosphate probes need only short 3′ OH probes for extension, reducing 3′ truncation synthesis. The combination of both polymerase and ligase discrimination increases specificity. In some aspects the detection sensitivity may be sufficient to eliminate the requirement for an amplification step.

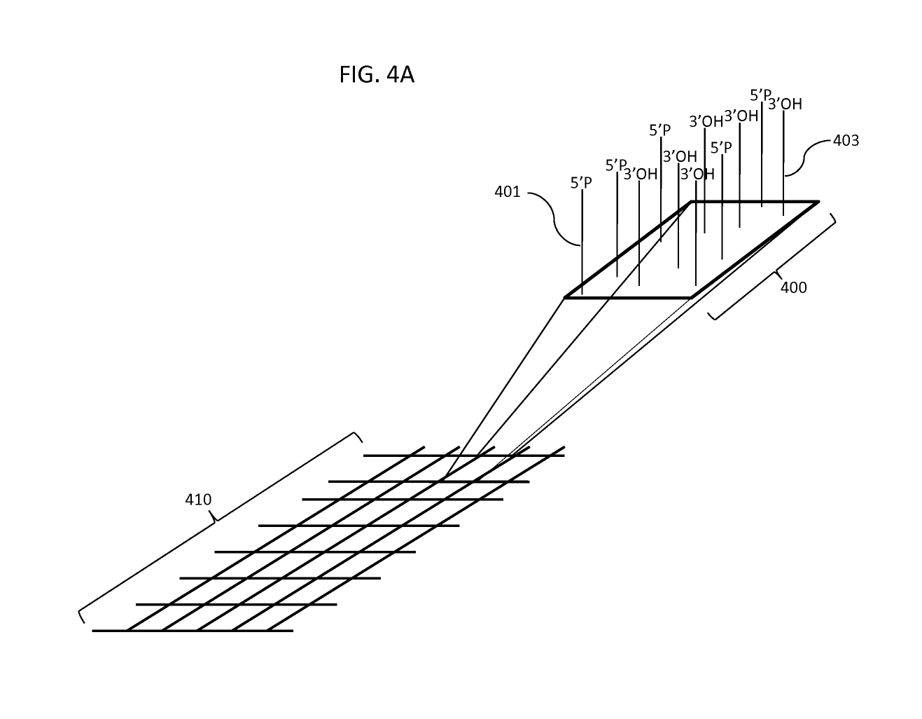

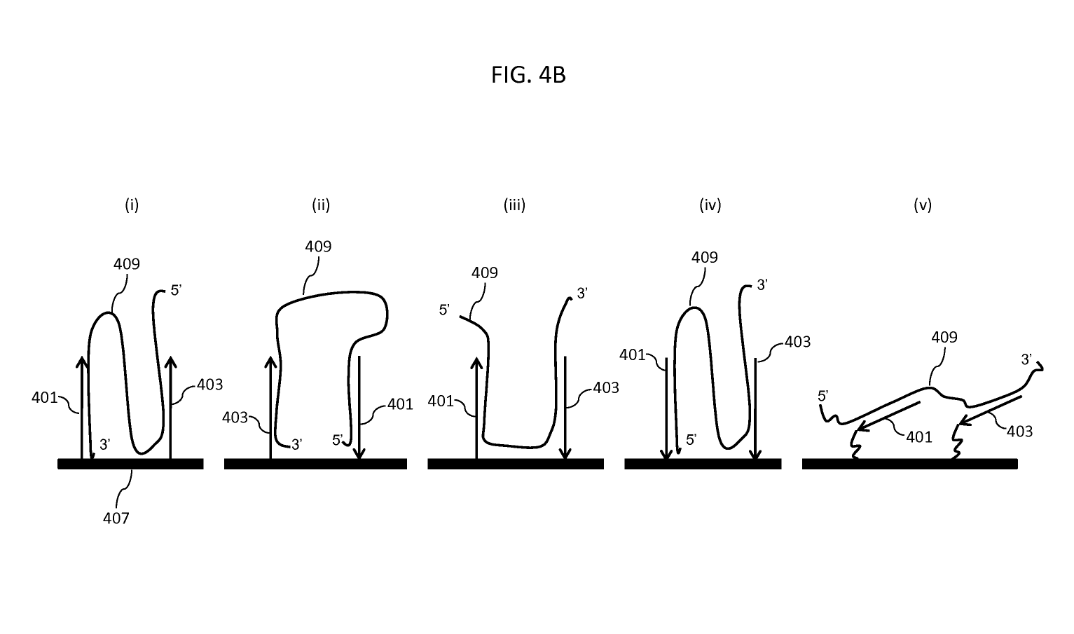

FIG. 4A illustrates schematically the arrangement of the two probes for each target in a dual probe embodiment. Each target feature 400 has a mixture of two probes, a first probe 401 that has a 5′ phosphate up orientation and a second probe 403 that has a 3′ hydroxyl up orientation. The probes are synthesized in the same region or feature 400 and may be arranged in an array 410 of features.

FIG. 4B shows alternative formats for the first probe 401 and the second probe 403 for a given target 409. Both probes may be 3′ up in relation to the support 407. Both probes can be extended using the hybridized target 409 as template. In some aspects the second probe 403 may be extended first with the first probe being blocked from extension by a protecting group and the protecting group can then be removed and the first probe extended. Alternatively, the first probe may be extended first followed by the second. In another aspect, shown in panel (ii) probe 403 is 3′ up and probe 402 is 5′ up. In another aspect, shown in panel (iii) probe 401 is 3′ up and probe 403 is 5′ up. In another aspect, probes 401 and 403 are 5′ up as shown in (iv) and (v). In panel (v) there are spacers at the 3′ ends of the probes that are not complementary to the target. The spacers extend the distance of the duplex from the array surface. In panel (iv) the probes 401 and 403 are complementary to the target over their lengths. Panels (i), (iv) and (v) may be referred to as “uni-polar format”. Panels (ii) and (iii) may be referred to as “bi-polar format”. Formates (iii) and (iv) may be expected to have steric limitations resulting from the required orientation of the region of the target that is between the regions hybridized to the array probes. This would be expected to vary depending on the length and sequence of the unhybridized central region.

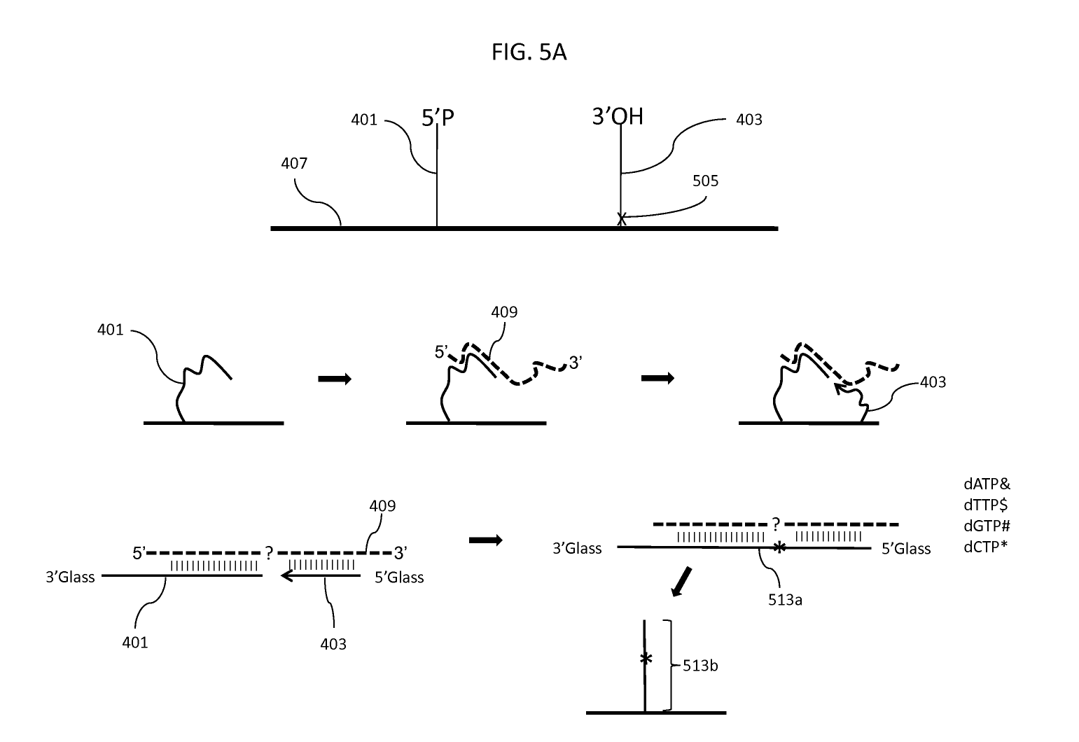

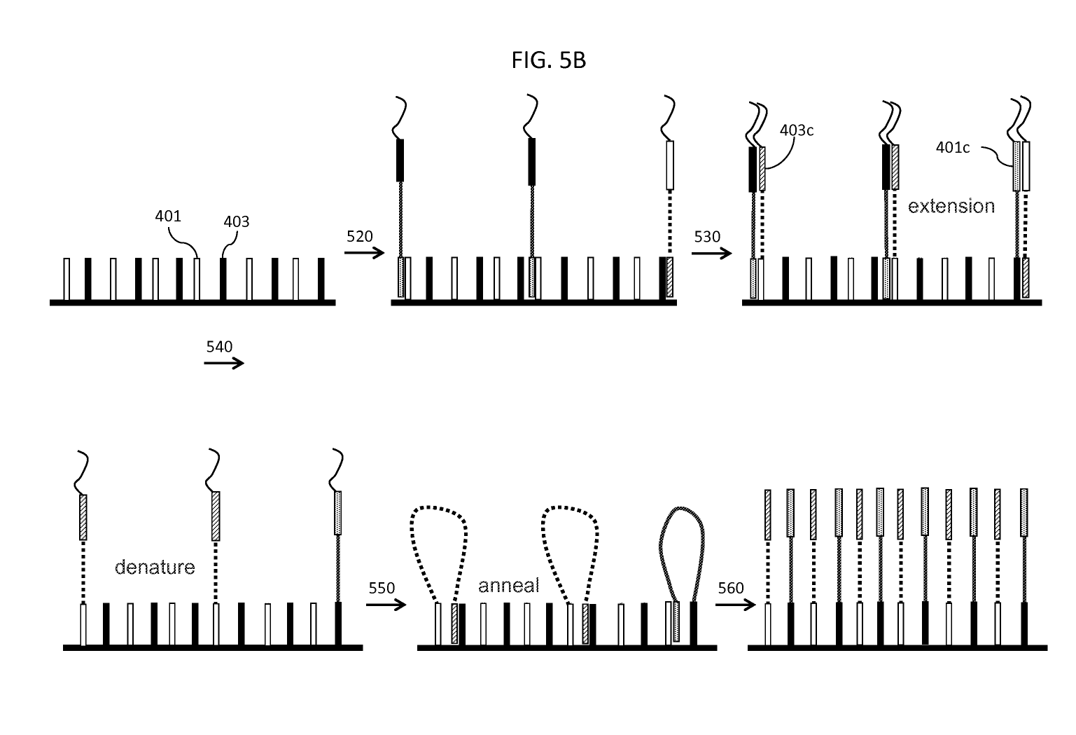

FIG. 5 shows a schematic of one embodiment of the HXLS assay. The features have two probe sequences, one being 5′ phosphate up 401 and the other being 3′ hydroxyl up 403 and having a cleavable linker 505 (e.g. a diol linkage) near the solid support 407. The probe that is 5′ phosphate up can hybridize to the target 509 to capture the target and then the 3′ up probe 403 binds to the captured target and can be extended at the 3′ end using the captured target as template. The 5′ up probe 401 may have a longer region of complementarity with the target thus binding with higher stability than the 3′ up probe which has a shorter region of complementarity with the target, at least initially (i.e. before extension). The hybridized target may have an unknown base to be sequenced, shown by a “?” in the lower panel. Following extension with a labeled base specific for the unknown base, the probes are ligated together to form a ligation product 513a with a labeled base in the center (*). The 3′ up probe may then be cleaved from the array using for example, aqueous sodium periodate, so that it is only attached if the extension and ligation steps have occurred resulting in a ligation product 513b that is now attached to the array at only one end. The incorporated label, (indicated by “*”) which may be a fluorophore, can then be detected. The assay uses hybridization for capture, specific priming by an array bound probe, providing polymerase specificity, followed by ligation, providing ligase specificity. The methods thus provide at least three levels of specificity: hybridization, extension and ligation.

FIG. 5B shows a schematic of a sequencing method. The array may contain forward 401 and reverse 403 primers that are both 3′ up on the array. The target hybridizes (step 520) to the forward primer and the forward primer is extended (step 530) to make a copy of the target. The copy is an extension of the forward primer so it is covalently attached to the array. The target strand can be separated by denaturation and washed away (step 540). The extension products have a region 403c that is complementary to the reverse primer 403 and region 401c that is complementary to forward primer 401. After extension and washing to remove the template strands the extension products can anneal to the opposite primer on the array (step 550) and those primers can be extended (step 560) using the extension product from step 530 as template. This can be repeated to generate amplified targets. This solid phase or bridge amplification has been previously described. In some aspects one of the array probes may be cleaved after amplification to remove the extension products from those primers from the array. This results in amplified products that are all the same strand rather than a population of extension product that are one strand and a population of extension products that are the complementary strand. The products can be sequenced using a generic sequencing primer.

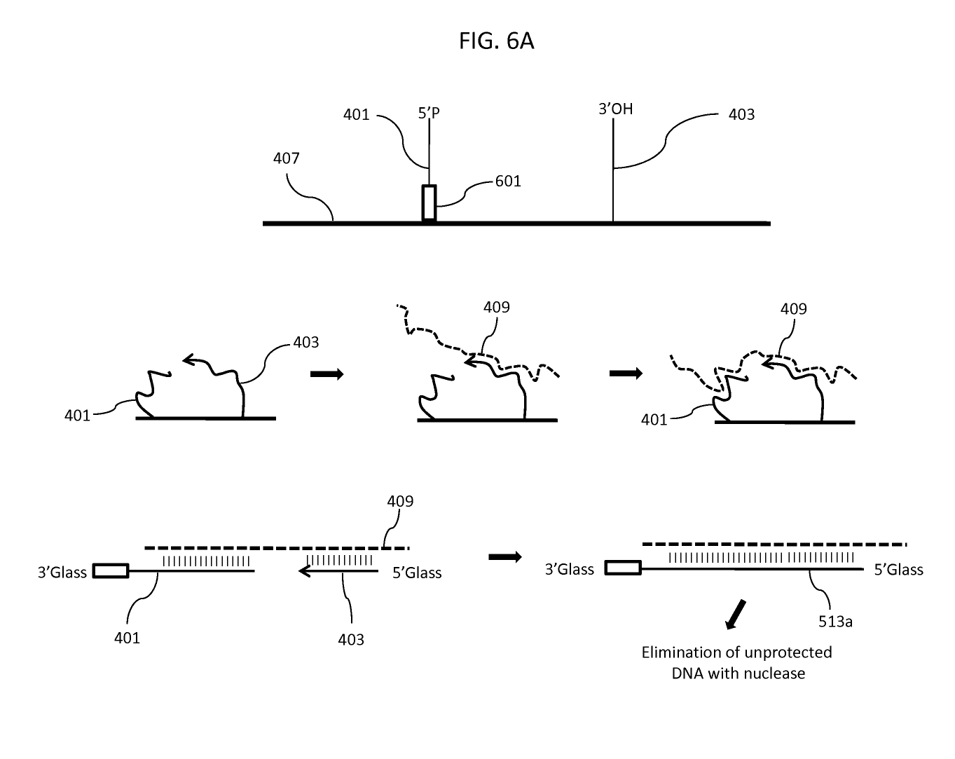

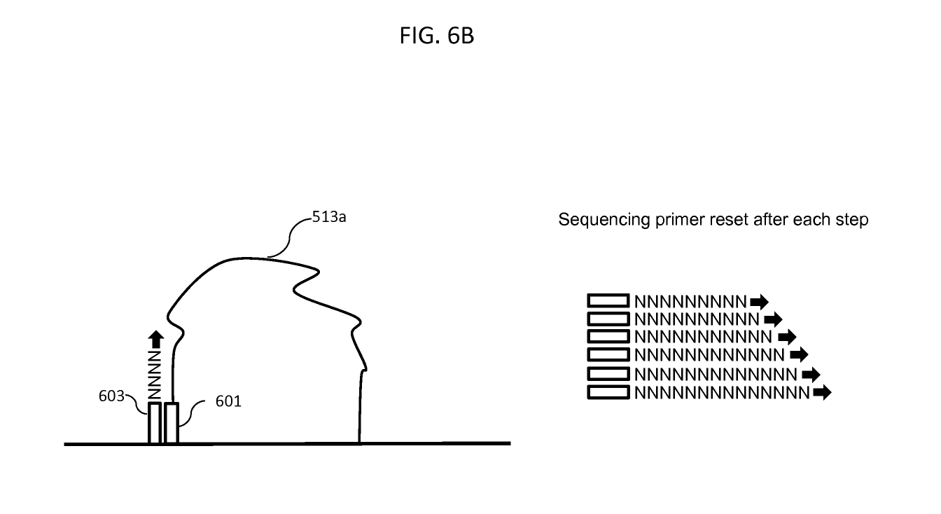

in another aspect, the methods may be used for on array target preparation for sequencing. An illustrative embodiment is shown in FIG. 6. The features have two probe types, one 5′ up and the other 3′ up as shown in FIG. 6A. The 5′ up probe has a universal priming site 601 proximal to the array surface 407. The target 509 is hybridized to the 3′ up probe 403 for capture and then to the 5′ up probe 401 for ligation. The 3′ up is extended using the target as template so a copy of the target is generated. The length of target that is copied corresponds to the length between the hybridization position on the target of the 5′ up probe and the 3′ up probe. The extension product is ligated to the 5′ up probe to form a ligation product. Unprotected probes and DNA can be digested with exonuclease. The ligation product is resistant because there are no free ends. The ligation product 513a can then be sequenced using the universal site 601 for binding of a sequencing primer 603 as shown in FIG. 6B. The primer may have a region that is complementary to the universal site and a degenerate region shown by N's. Successive rounds of hybridization and single base extension and detection using primers of increasing length can be used. After each step of sequencing to determine the next base the primer can be reset using a primer having a length of degenerate region that is 1 base greater than the last primer. Methods for sequencing using degenerate primers are discussed, for example, in Tang et al. J. Genet. Genomics (2008), 35:545-551.

In some aspects the methods are combined with method of nucleic acid analysis. The methods may be used in connection with methods for SNP genotyping, including single base extension (SBE) and minisequencing methods such as those disclosed in Shapero et al. Genome Res. 11:1926-1934 (2001). Methods for genotyping SNPs include, for example, multiplex minisequencing using tag-arrays as disclosed in Milani and Syvanen, Methods Mol boil 2009, 529:215-229. Methods for bridge amplification are disclosed in U.S. Pat. No. 6,300,070 and in Bing et al. 1996, “Bridge amplification: a solid phase PCR system for the amplification and detection of allelic differences in single copy genes”, in the proceedings of the Promega 7^{th} international symposium on human identification. In another aspect the methods are combined with methods for anchored multiplex amplification on a microelectronic array as described in Westin et al. Nature Biotech. 18, 199-204 (2000). Briefly, template is captured by hybridization to a support bound strand displacement amplification (SDA) primer. The SDA primer is extended and subsequent rounds of extension and strand displacement from a nick generated at a BsoB1 nicking site result in multiple copies of the complement of the target attached to the solid support. Another SDA amplification method that may be used in combination with the presently disclosed methods is described in Walker et al. PNAS 89:392-396 (1992). Briefly, the method uses restriction enzyme cleavage and heat denaturation of the DNA sample to generate two single stranded target fragments. Two amplification primers bind to the targets resulting in a 5′ overhang of the primers. The overhang has a restriction site for HincII. The target is extended using the primer as template to make the HincII site double stranded. The extension incorporates phosphorothioate into the target strand to generate a hemiphosphorothiolated HincII site which is subsequently used for nicking in the primer. The nick site is extended using the target as template and displacing the previous primer extension product. The HincII site is regenerated each time the primer is extended so it can be repeated.

Synthesis of two distinct probes in the same feature space is possible via various methods. An exemplary synthesis strategy may be as follows: (1) couple C-start on a Bisb wafer and photolyze mask pattern; (2) couple 1:1 MP-PEG+DMT-PEG amidite mixture and photolyze mask pattern; (3) synthesize first probe with 5′ or 3′-NNPOC and cap terminal hydroxyl groups with capA/capB; (4) detritylate with TCA in flowcell; (5) synthesize second probe with 5′ or 3′-NNPOC; (6) standard open square photolysis; and (7) standard deprotection and packaging. Methods for synthesis and photocleavable protecting groups are disclosed, for example, in U.S. Pat. Nos. 7,144,700, 7,087,732, 6,833,450, 6,8010,439 and 6,800,439, which are each incorporated herein by reference in their entireties. See also U.S. Pat. Nos. 6,566,495 and 6,506,558, also incorporated by reference in their entireties. (PEG in this context refers to polyethylene glycol).

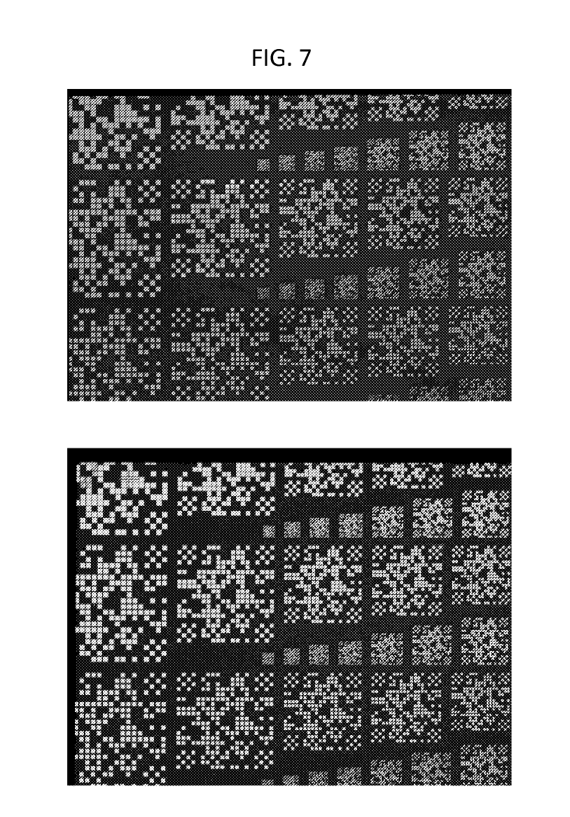

Both probes can be synthesized using this approach. FIG. 7 demonstrates that two different probe sequences can be synthesized simultaneously in the same features. The array was synthesized to have two different probe sequences in a plurality of the features and the hybridization pattern demonstrates that both probes are correctly synthesized and detectable by hybridization. The features were synthesized with “probe 1”, which is 5′ tggaggattt aacccaggag ag 3′ (SEQ ID No. 1) and “probe 2”, which is 5′ tatcatggtc actgggtagg tg 3′ (SEQ ID No. 2). Both sequences should be present in each of the features. This was tested by separately hybridizing the arrays to biotin labeled probes that were either complementary to probe 1, 3′ acctcctaaa ttgggtcctc tc-biotin 5′ (SEQ ID No. 3), hybridization pattern shown in the upper panel, or complementary to probe 2, 3′ atagtaccag tgacccatcc ac-biotin 5′ (SEQ ID No. 4), hybridization pattern shown in the lower panel.

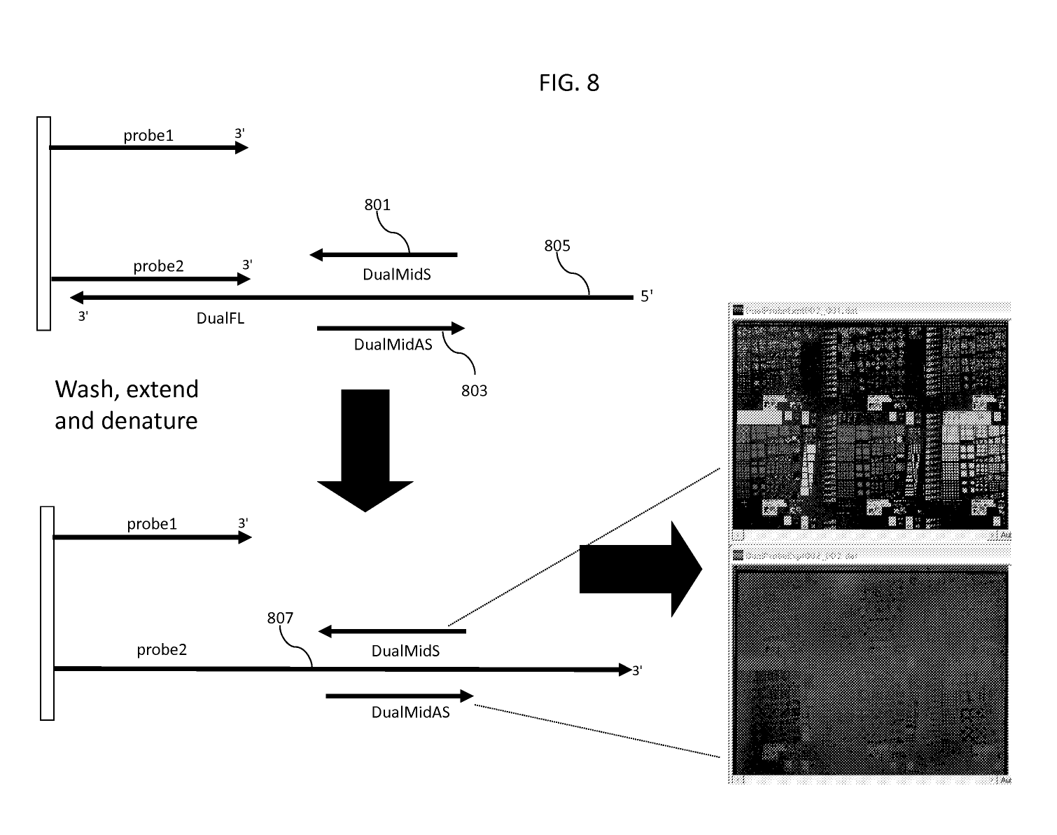

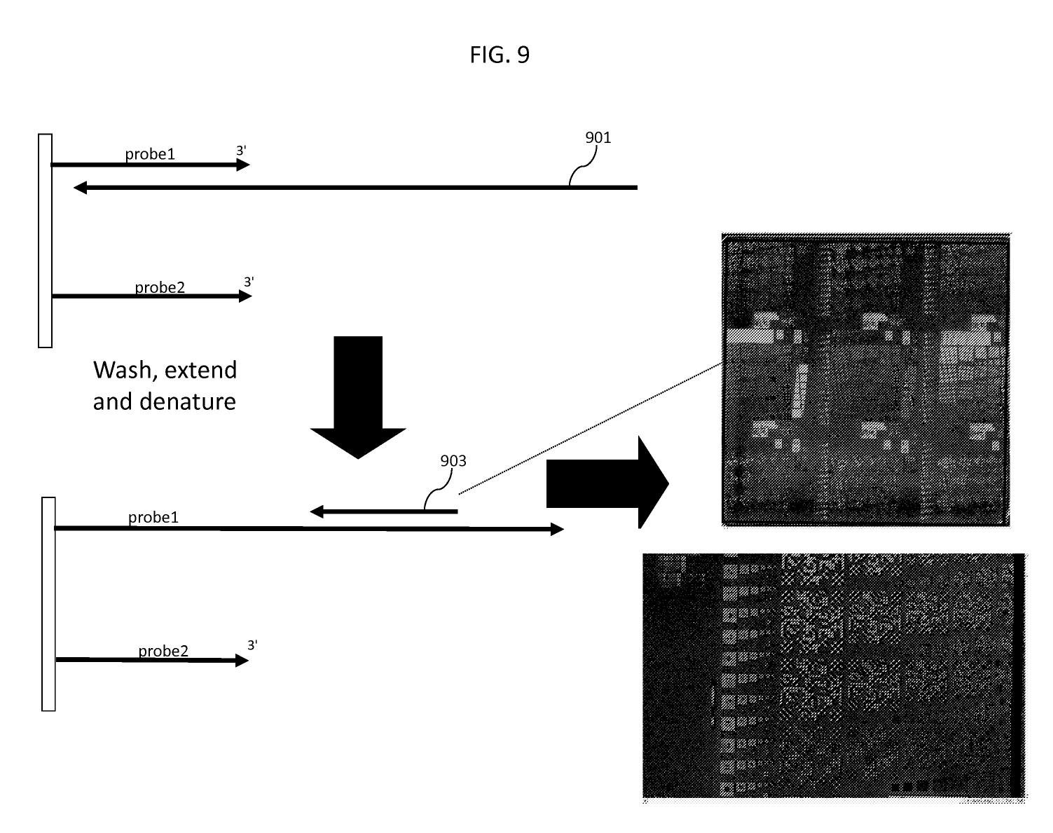

Preferably 3′ up probes synthesized in a dual synthesis are capable of base extension by DNA polymerase. This activity is demonstrated in FIGS. 8 and 9. FIG. 8 shows schematically the hybridization of DualFL oligo 805 (120 nt) to probe 2. The DualMid “S” 801 (sense) or DualMid “AS” 803 (antisense) are also shown. The DualFL oligo 805, was first hybridized to the array, then washed, extended and denatured to remove the DualFL oligo 805. Probe 2 was extended using DualFL 805 as template to make extension product 807. Then the DualMidS 801 and DualMidAS 803 oligos were then hybridized. The DualMidS 801 probe should hybridize but the DualMidAS 803 should not since it has the same sequence as the extension product. The hybridization pattern images are shown on the right for the DualMidS (upper) and DualMidAS (lower). As expected, hybridization is observed for DualMidS but not DualMidAS, demonstrating that the hybridization and extension step are functioning as expected.

FIG. 9 is similar to FIG. 8, but the target used for hybridization is complementary to probe 1. The DualFLcomp oligonucleotide probe 901 is 120 nucleotides and hybridizes to probe 1 and probe 1 is extended using 901 as template. The DualMidAS probe 903 is complementary to the extension product and is labeled. Hybridization of the DualMidAS is shown on the right.

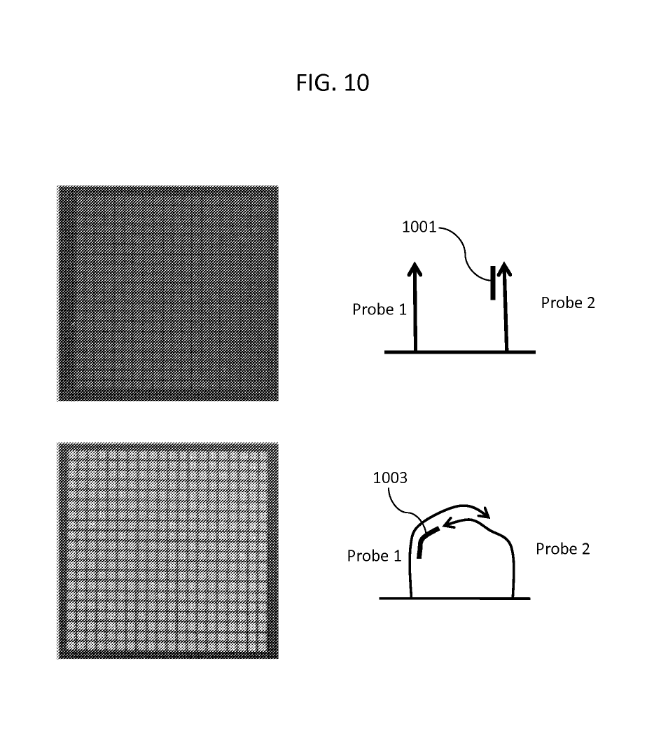

In some aspects it is also desirable that the two probes are capable of bridging together for polymerase and ligase activities. The experiment shown in FIG. 10 demonstrates that such bridging can occur between the 2 probes, and that DNA ligase is active under such conditions. FIG. 10 shows fluorescence scans on the left and schematics of the assay on the right. In the upper panel on the left the labeled reporter 1001 that is used in the upper panel hybridizes to the 3′ end of probe 2 and blocks hybridization of the 3′ end of probe 2 to the 3′ end of probe 1. In the lower panel, the labeled reporter 1003 hybridizes to probe 1 immediately adjacent to where the 3′ end of probe 2 can hybridize to probe 1. The reporter 1003 can then be ligated to the 3′ end of probe 2 only in the presence of probe bridging. The presence of signal in the hybridization scan shown at the lower left demonstrates the bridging of probes 1 and 2. The upper panel on the left shows background levels of signal, demonstrating failure of the labeled probe 1001 to ligate to the probes on the array.

In preferred aspects an array may be designed to capture and assay selected groups of target sequences, for example, a collection of coding exons or all coding exons. Each coding exon would be targeted by at least one feature, each feature having two probe sequences, a 5′ up and a 3′ up probe. The 5′ up probe defines one end of the target exon and the 3′ up probe defines the other end of the target exon sequence to be amplified. Longer exons may be targeted by more features so that sequencing can be initiated from a number of regions within the exon or the target to be sequenced.

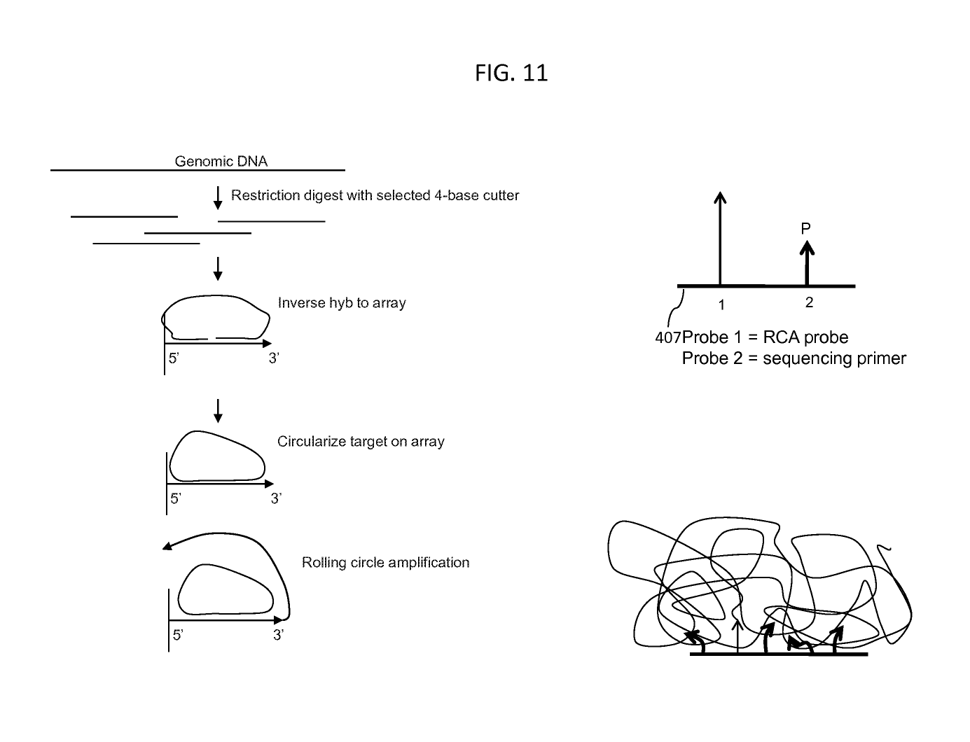

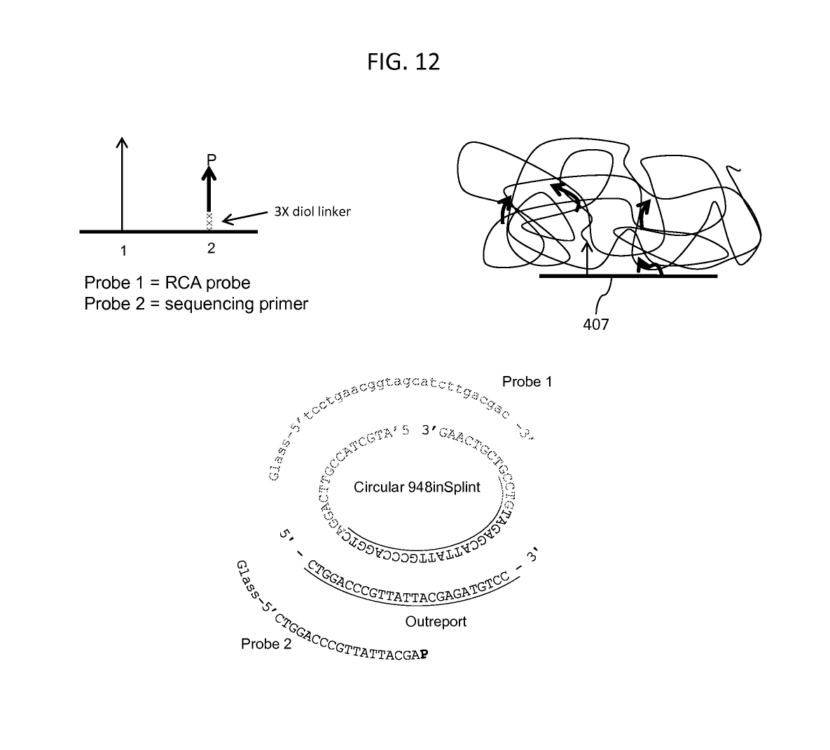

In another aspect illustrated in FIGS. 11 and 12 the dual probe method is combined with rolling circle amplification. As shown on the left side of FIG. 11, the genomic DNA is fragmented, for example, using shearing, sonication or a restriction enzyme or combination of restriction enzymes, the fragments are hybridized to array probes that splint the ends together. The ends are joined to form a circle and the probe is extended using the circle as template and resulting in a RCA product. The RCA product is tethered to the support. Sequencing primers for the target are also included in the vicinity, preferably as part of the same feature. The sequencing primer can be extended by ligation, single base or any other sequencing method. Similarly, in FIG. 12 the sequencing primer is present in the same feature but is released by cleavage after the RCA product has been generated. The localized concentration of the sequencing primer is higher in the immediate vicinity of the feature and the probability of hybridization to the RCA product is increased. Sequencing can be by any method of extension of the sequencing primer, e.g. ligation or single base extension.

Related methods are disclosed in U.S. patent application Ser. No. 12/899,540 which is incorporated herein by reference in its entirety. The fragments are denatured to obtain single strands that are hybridized to probes on the solid support. The probes are designed to be complementary to the ends of restriction fragments of interest and to hybridize to those targets so that the ends of the targets can be circularized as shown. The 3′ end of the target may be extended by polymerase to bring the ends in proximity for ligation if needed. The circularized target is used as template for RCA. The second probe may then be used as a sequencing primer. In another aspect, shown in FIG. 12 the second probe has a cleavable linker group and can be cleaved from the array after RCA reaction. The released probe 2 may serve as a sequencing primer for the RCA product. In some aspects the cleavable linker is 1-3 diols.

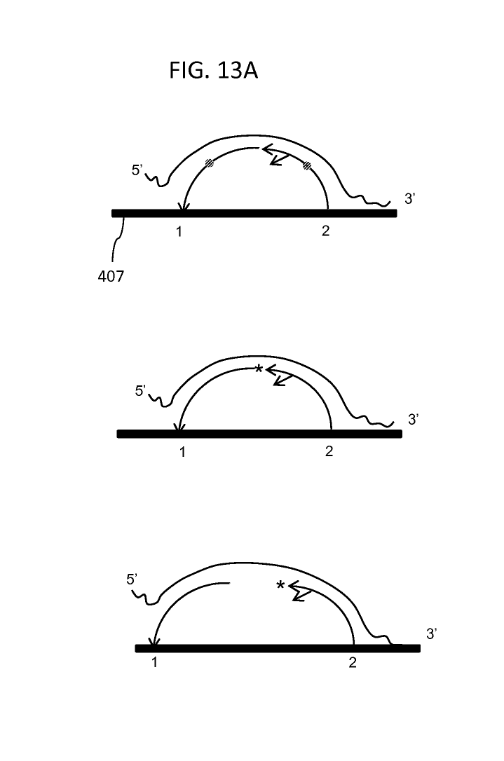

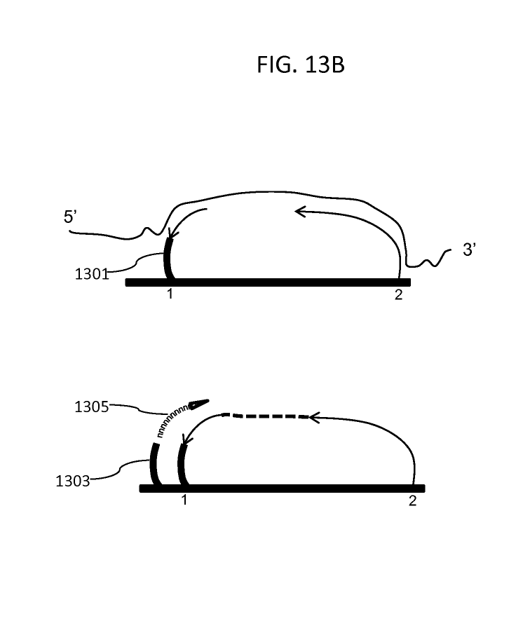

FIG. 13A shows schematics for using the method for allele specific detection of SNPs and copy number. Probes 1 and 2 are complementary to a selected target and hybridized to the target with either no gap (top), a single base gap (middle) or a larger gap (bottom). For SNP genotyping the SNP may be within either probe 1 or probe 2 (see the closed circles as examples), but more preferably it is at the 3′ end of probe 2 or 5′ end of probe 1 so that if the non-complementary base is present the ligation will be inefficient. Allele specific discrimination at the ligation step may be used to determine which alleles of the SNP are present. In the middle panel the SNP may be at the gap position so that the base that is added can be used to determine which alleles are present. The SNP may also be positioned at the 3′ end of probe 2, and extension may be allele specific, or the 5′ end of probe 1, and ligation may be allele specific. In these embodiments ligase is preferably used to join the ends of probes 1 and 2, making them resistant to exonuclease. Exonuclease can then be used to remove probes that are not ligated. The In the center panel there is a single nucleotide gap between the first and second probes when hybridized to the target. Probe 2 is extended by a single base followed by ligation. The probes are treated with exonuclease to digest probes that are not ligated. Similarly, in the lower panel the second probe is extended through a larger gap, more than 1 nucleotide, followed by ligation and treatment with exonuclease. The lengths of probes 1 and 2 can be varies to improve sensitivity and specificity. For example, probe 1 may be longer than probe 2 or probe 2 longer than probe 1.

FIG. 13B shows methods for sequencing using pairs of co-located probes 1 and 2. The 3′ up probe 2 can be extended using the hybridized target as template. The extension product (dashed line) is ligated to the end of probe 1 which also contains a generic region 1301 for hybridization of a sequencing primer. After ligation the support can be subjected to exonuclease treatment. The sequencing primer has a region 1303 that is complementary to the generic region 1301 and a degenerate region 1305 to hybridize to the target specific region of probe 1. The degenerate region hybridizes to the target specific region of probe 1 and can be used for sequencing using any extension based method. Sequencing may, for example, include extension with acyclic nucleotides or reversible terminators. In some aspects multiple rounds of extension and detection followed by removal and resetting of the primer may be used.

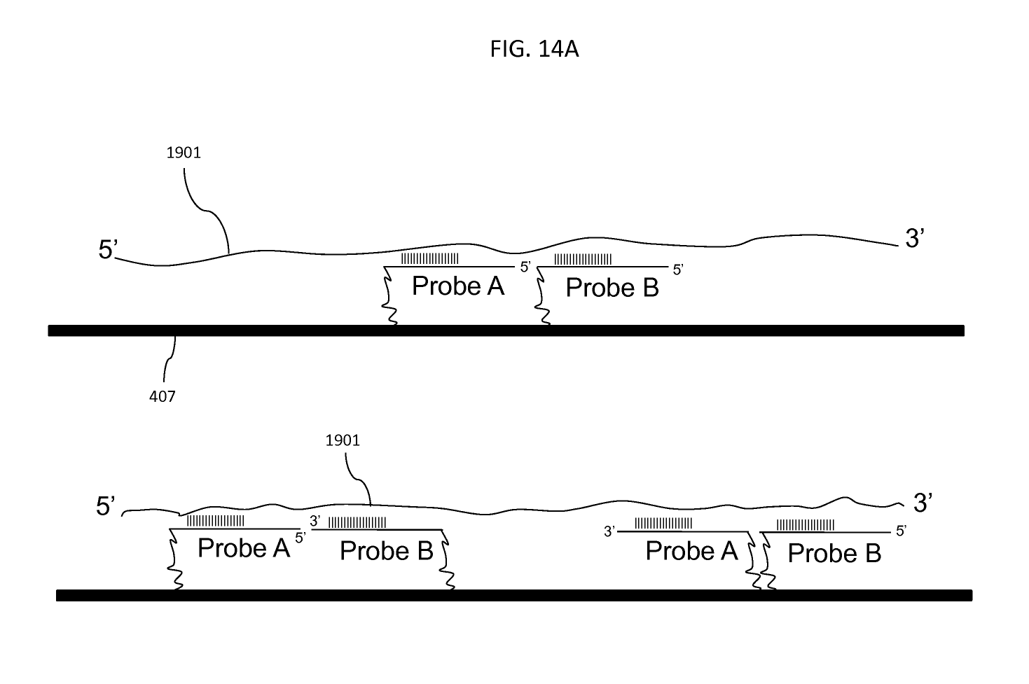

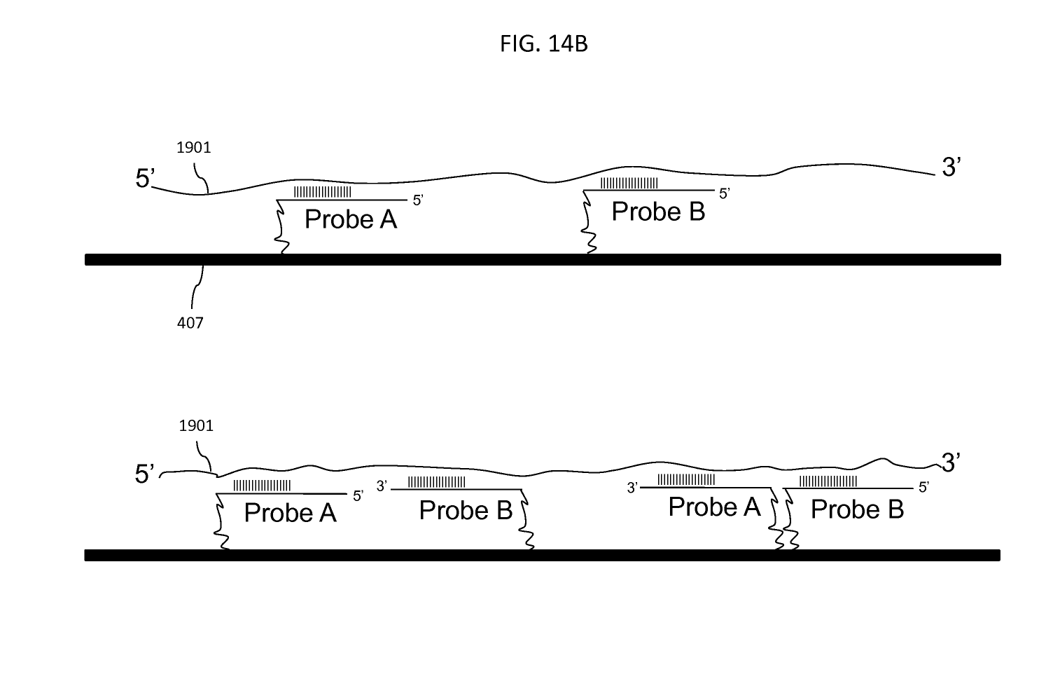

FIG. 14A shows different methods for using two probes to capture labeled DNA targets 1901. The probes shown have a spacer region shown by the vertical portions of the probes and a target specific portion shown by the horizontal portions of the probes. The probes may be both 5′ up as shown in the upper panel or both 3′ up. Alternatively one probe can be 5′ up and the other 3′ up and they can be arranged so the ends are directed toward one another when hybridized to target as shown in the bottom left or directed away from one another as shown in bottom right. The two probe system provides for cooperative binding such that the two probe complex is more stable than the individual complexes combined. In some aspects a SNP may be positioned in one of the probes. The SNP may be in the middle or a probe or at the end of a probe. In preferred aspects there are no gap positions between the two probes, probe A and probe B, when they are hybridized to the target.

In another aspect shown in FIG. 14B there are gaps between the two probes, probe A and probe B, when hybridized to the target. The probe configurations are as described for FIG. 14A, but there is a gap of one or more bases between the probes when hybridized to the target. The gap may be, for example, 1 to 20 or 30 bases. In some aspects the gap may be 30 to 100 bases or more. The presence of the gap alters the cooperative binding nature of the probes.

### EXAMPLES

### Example 1

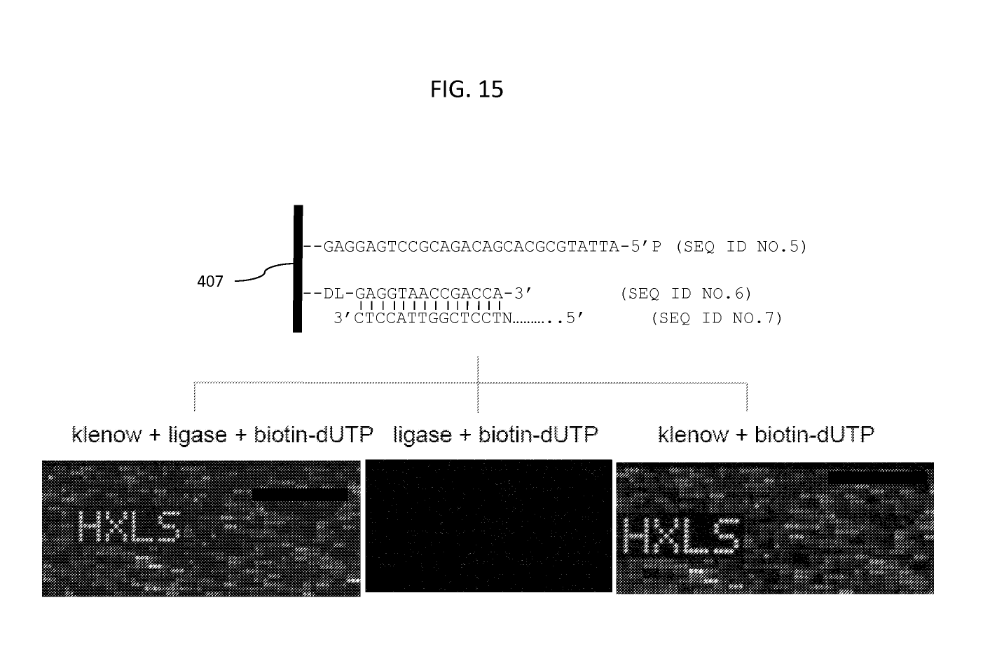

Demonstrating templated polymerase extension of a 3′ up probe. In FIG. 15 polymerase extension on a 3′ up probe is shown. Features having both the 5′ up probe and the 3′ up probe are arranged in a pattern that spells “HXLS”. The features have a first probe that is 5′ up (3-GAGGAGTCCG CAGACAGCAC GACTATTTA-5′ (SEQ ID No. 5)) and a second shorter probe that is 3′ up (5′GAGGTAACCG ACCA-3′ (SEQ ID No. 6)). A solution probe (SEQ ID No. 7: 5′ CTCCATTGGCTCCTN . . . -5′) that is complementary to the 3′ up probe is hybridized to the array and then treated with klenow, ligase and biotin-dUTP (left), with ligase and biotin-dUTP (center) or with klenow and biotin-dUTP (right). As expected in the presence of klenow the biotin-dUTP is covalently attached to the array probes in a sequence specific manner showing that the 3′ end probe is available for extension.

### Example 2

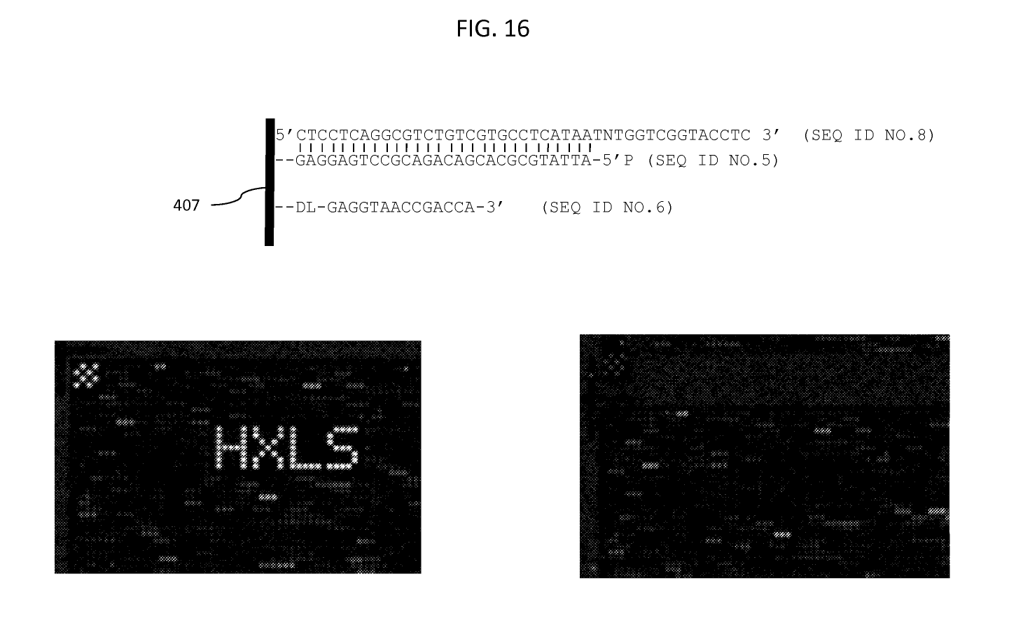

Demonstrating ligation of a labeled oligo to the 5′ end of a 5′ up probe. FIG. 16 shows the results of ligation of a labeled probe to the 5′ end of a probe terminating with a 5′ phosphate. A schematic is shown in the upper portion. The features have the same array probes as FIG. 1S. A solution probe (5′ CTCCTCAGGC GTCTGTCGTG CTCATAATNT GGTCGGTACC TC-3′) (SEQ ID No. 8) is hybridized to the array on the left and hybe buffer alone is added on the right. The array is subjected to a stringency wash and a 5′ Biotin-NNNNNNNNN-3′ probe is added along with ligase followed by a high stringency wash. If the biotinylated probe is ligated to the 5′ up probe the feature will be labeled. The features that have the complementary probe are arranged in the shape of the letters “HXLS” and light up on the image on the left as expected, but not on the right.

### Example 3

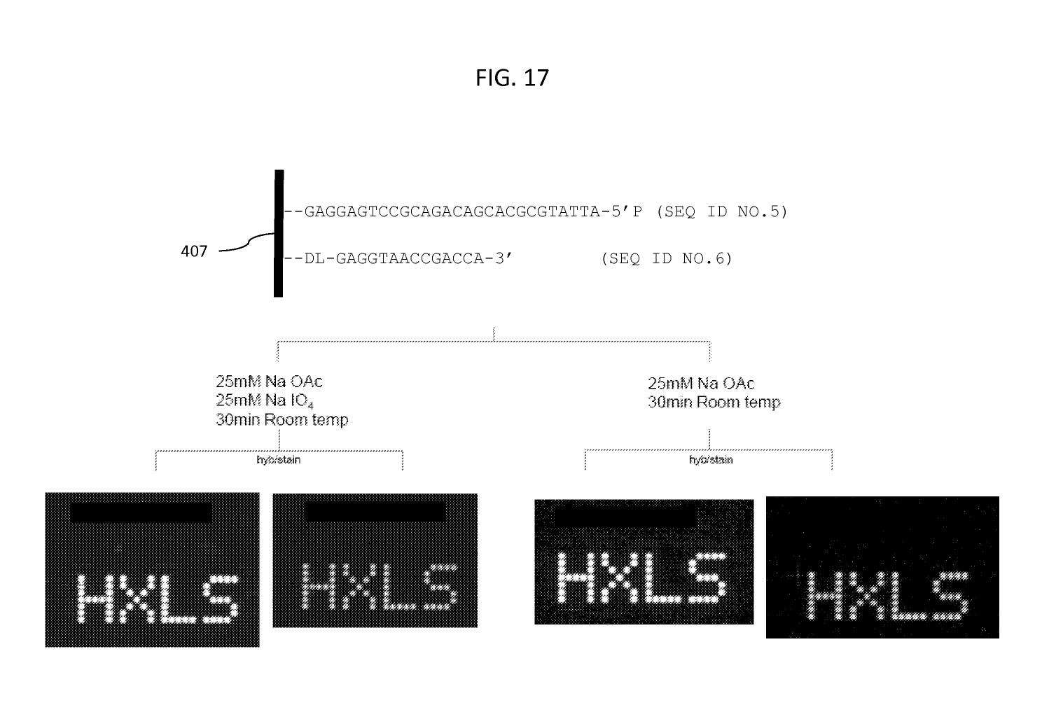

Cleavage of the 3′ up probe from the array. Using an array having the probe sequences as discussed above in reference to FIG. 10 a test of the diol-linker cleavage was performed. The conditions tested for cleavage were either 25 mM NaOAc, 25 mM NaIO₄ and 30 min at room temp or 25 mM NaOAc at room temp for 30 min. After the treatment the arrays were hybridized to either a 3′ biotinylated probe complementary to the 5′ up probe 5′ CTCCTCAGGCGTCTGTCGTGCTCATAAT 3′ SEQ ID NO. 15 (1^{st} and 3^{rd} from the left) or a probe complementary to the 3′ up probe 5′ TGGTCGGTACCTCAA SEQ ID NO. 16 (2^{nd} and 4^{th}). The results are shown in FIG. 17. Cleavage reduced the signal from the 3′ up probe in both conditions (65,000 vs. 25,000 and 65,000 vs. 30,000) but there is still significant signal from the 3′ up probe suggesting that the diol linkage cleavage is not complete but roughly 50%.

### Example 4

Cleavage of the 3′ up probe using multiple diol linkers. In a subsequent experiment 3′ up probes were synthesized with 1 or 3 diol linkers in single or dual synthesis and subjected to cleavage with 0, 25, 50 or 100 mM NaIO₄ for 30 min at room temp and then hybridized with a fluorescently labeled oligonucleotide complementary to the 3′ up probe. The probes were 22 mers. The reduction in intensity was quantified and is shown in Table 1. The conditions tested were: A 100//-PEG-(DL)₁-probe#1b (5′-3′) “1 diol-single”; A100//-PEG-(DL)₃-probe#1 (5′-3′) “3 diol-single”; or A100//-PEG-(DL)₃-probe#1 (5′-3′) with -PEG-probe#2 (5′-3′) “3 diol-dual”. The use of 3 diols significantly improved the cleavage in both single and dual probe synthesis.

TABLE 1

| | 0 mM NaIO₄ | 25 mM | 50 mM | 100 mM |
|---|---|---|---|---|
| 1 diol-single | 0 | 53% | 71% | 64% |
| 3 diol-single | 0 | 96% | 96% | 95% |
| 3 diol-dual | 0 | 85% | 82% | 85% |

### Example 5

In another example, the dual probe array features were tested for hybridization of a target oligonucleotide, extension and ligation and then subjected to exonuclease treatment. As discussed above, the features that have the dual probes were arranged in the pattern of “HXLS” so the expected result was a scan image having the pattern of the “HXLS” letters detectable and the remainder of the array showing background levels of signal. Three conditions were tested. The first conditions was without exonuclease was used and as expected the background signal observed was high and the HXLS signal was high has well, ˜36,000. For the second condistion, 20 U of Exo I and 200 U of Exo III were used and as expected the background signal was faint (the image appears black) and the HXLS pattern can be seen clearly although it is fainter than in the first condition (signal ˜1900). For the third condition 60 U of Exo I and 600 U of Exo III were used and the results were similar to the second condition, very low background and signal ˜1600. This demonstrates that exonuclease reduces background.

### Example 6

Testing different polymerases. In another example different polymerases were tested. The enzymes tested were, (1) Klenow exo-, (2) T7 DNA polymerase, (3) AMPLITAQ Stoeffel fragment, and (4) T4 DNA polymerase. Each of the polymerases gave the expected pattern, with the AMPLITAQ Stoeffel fragment and Klenow exo- giving the highest signal (1100 and 3200 respectively) and the T7 DNA polymerase giving the lowest signal (270). The signal for T4 DNA polymerase was 850.

### Example 7

In another example, the ability of the polymerase to discriminate between addition of the proper base and addition of non-cognate bases was assayed. Eight different arrays were processed, one for each of the four expected bases using either FAM G&C or Biotin A&T. The observed per feature signal for the features in the pattern are provided in Table 2. As expected where C or G are expected the highest signal is obtained when FAM-G&C is present (600 and 4800 signal). When Biotin-A&T are present highest signal was observed for the array where A is expected (1100) but the C, T and G have very similar signal.

TABLE 2

| | FAM-G&C | Biotin-A&T |
|---|---|---|
| C expected | 600 | 120 |
| T expected | N/A | 220 |
| A expected | 150 | 1100 |
| G expected | 4800 | 280 |

### Example 8

In another example whole genomic DNA was hybridized to arrays of dual feature probes for selected targets. A human placental DNA sample was fragmented and hybridized to the array. Each test marker on the array has a 5′ up probe and a 3′ up probe corresponding to a site on a selected the genomic target so that there is a single base gap between the ends of the two array probes when they are hybridized to the target. Following a stringency wash, a mixture of biotin-dATP, biotin-dUTP, FAM-dCTP and FAM-dGTP was used to extend the 3′ up probe in a gap fill reaction. In the presence of DNA ligase, the two probes can be covalently joined together to seal the filled gap. The array was then treated with exonuclease to digest any unligated 3′ up probes. Biotin and FAM detection was performed similar to the Affymetrix AXIOM assay and analysis revealed if the identity of the labeled nucleotide used to fill the gap corresponds to the sequence of the hybridized genomic template.

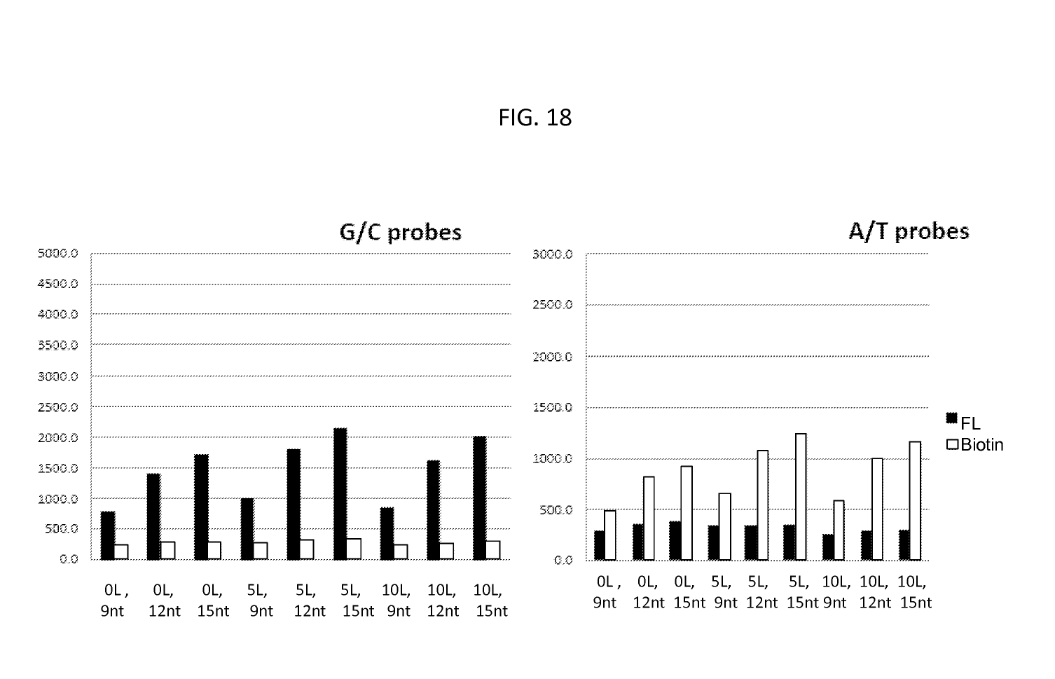

The results are plotted in FIG. 18 according to the length of the 3′up probe and the length of the linker on that probe. The graph on the left shows raw signal for G/C probes in either the FL channel (FAM) or the biotin channel. The graph on the right is raw signal for A/T probes in either the FL channel (FAM) or the biotin channel. In both graphs the FL channel is shown by filled bars and the biotin channel is shown by open bars. As expected, the signal for the G/C probes is primarily in the FL channel (graph on left) and the signal for A/T probes is primarily in the biotin channel (graph on right). The different conditions shown are linker length (0, 5 or 10 MP-PEGs) and the length of the 3′ up probe: 9, 12 or 15 nucleotides. For both the G/C and A/T probes the highest signal was observed with a linker length of 5 and a 3′ up probe length of 15 nt.

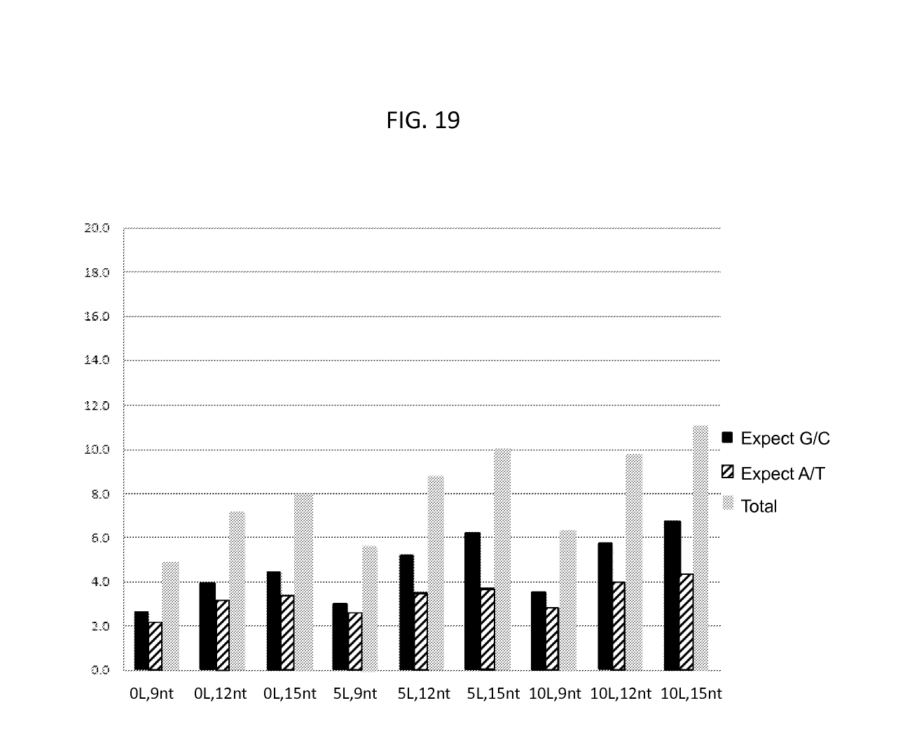

Shown in FIG. 19 is a bar graph of signal intensity from different targets separated into those targets that are expected to incorporate a G or C and those that are expected to incorporate an A or U labeled nucleotide. The total is also plotted. The results are also grouped by length of the linker (0, 5 or 10 MP-PEGs) and length of the 3′ up probe (9, 12, or 15 nt).

Probes were sorted into bins by the last base in the 5′ up probes or the last base in the 3′ up probes compared to the assay base in either the GC or AT channels. For the 5′ up probes the G and C assay base gave the greatest signal in the GC channel and the A and T assay base gave the greatest signal in the AT channel. For the 3′ up probes The G assay base and the A assay base gave the most consistent results.

To test the impact of the last base in the 5′ up probe or the last base in the 3′ up probe, the probes of the array were sorted by their last base and by the expected assay base then plotted by signal in either the GC channel or the AT channel. The specificity and sensitivity of the assay were not dependent on either the last or the second to last bases in the probes suggesting that truncation of the probes does not contribute to background. Truncation of 3′ OH probes was not detected.

The arrays can be made with co-synthesis of 5′ P and 3′ OH probes.

### Example 9

Extension from an arrayed template was tested. The array probe was 3′ TATGACCCGATAGCGTTGTGTTGGTGGAGACGGCT-5′ (SEQ ID No. 9) attached to the support at the 3′ end. A 5′ FAM-TATCGCAACACAACCACCTCT-3′ (SEQ ID No. 10) oligo which hybridizes to the underlined region of the arrayed probe was hybridized to the array and subjected to extension in the presence of either labeled ddGTP, ddCTP, ddUTP or ddATP. The perfect match to the next base in the array probe sequence is G and as expected the signal for ddGTP is highest, 25,000 counts. The signal for U is 600 counts, for C is 7,000 counts and for A is 4,500 counts.

In some aspects it may be preferable to use a polymerase that has a proofreading function. Methods for single base extension (SBE) using proofreading polymerases and phosphorothioate primers have been disclosed in, for example, Di Giusto and King, NAR (2003), 31(3):e7. In the absence of a proof reading function mis-incorporation can be high. In a test of the assay for discrimination using either Klenow or Klenow Exo- with a G expected as the perfect match (PM) the discrimination from the mismatch bases (MM) is better when Klenow exo- is used (see Table 3).

TABLE 3

| | Klenow | Klenow exo- |
|---|---|---|
| G (PM) | 7,000 | 8,500 |
| A (MM) | 2,700 | 450 |
| U (MM) | 250 | 100 |
| C (MM) | 2,200 | 1,500 |

### Example 10

Enzyme titrations were tested to determine if this improved fidelity. The enzyme concentrations tested were 0.04 U/μl, 0.01 U/μl, 0.004 U/μl and 0.01 U/μl plus SSB. The enzyme in this experiment was THERMOSEQUENASE (USB). The probe on the array was 3′ TATGACCCGATAGCGTTGTGTTGGTGGAGACGGCT-5′ (SEQ ID No. 11) and the solution probe was 5′-FAM-TATCGCAACACAACCACCTCT-3′ (SEQ ID No. 12). The solution probe was added at 25 mM in wash A for 30 min at room temp then washed in 0.2× wash at 37° C. for 30 min. The extension was in 1× thermoseq buffer, (260 mM Tris pH 9.5 and 65 mM MgCl₂ at 45° C. for 15 min in a 100 μl volume. For each condition there were 4 separate reactions each having 1.5 μl of 10 μm biotin-ddNTP (either G, A, U or C). Different dilutions of enzyme at 4 μg·μl were added and for the reactions with SSB, 1.5 μl of epicenter SSB 2 μg/μl was added to each of the 4 reactions. After incubation the arrays were rinsed with wash A and stained with SAPE for 15 min, scanned at 570, 0.2 laser, 500 pmt. The highest signal and best discrimination was observed at the lowest enzyme concentration. The top row of Table 4 shows the different enzyme concentrations. The results for the addition of SSB are shown in the last column.

TABLE 4

| | 0.4 μg/μl | 0.01 μg/μl | 0.004 μg/μl | 0.01 μg/μl. + SSB |
|---|---|---|---|---|
| G (PM) | 6500 | 9500 | 13000 | 9500 |
| A (MM) | 1700 | 400 | 350 | 150 |
| U (MM) | 300 | 150 | N/A | 100 |
| C (MM) | 4000 | 3500 | 500 | 1000 |

### Example 10

To test RCA from probe 1 followed by sequencing from probe 2 with or without release of probe 2. Probe 1 was: Glass-5′tcctgaacggtagcatcttgacgac-3′ and probe 2 was: Glass-5′ [Cleavable linker]-[Cleavable linker]-[Cleavable linker]-ctggacccgttattacga-3′P. Probe 2 is phosphorylated at the 3′ end to block extension. The ability of probe 1 to prime RCA given a circularized template was tested and confirmed. Probe 2 was tested and found to require dephosphorylation prior to extension as expected. Probe 1 RCA followed by probe 2 extension was tested with cleavage before or after dephosphorylation of probe 2. Probe 2 extension from the probe 1 RCA product was observed in both conditions but cleavage after dephosphorylation gave a 10 fold stronger signal. Circular 948inSplint is

(SEQ ID NO. 13)
3′GAACTGCTGCCTGTAGAGCATTATTGCCCAGGTCAGGACT
TGCCATCGTA′5
and
“outreport is”
(SEQ ID NO. 14)
-5′ CTGGACCCGTTATTACGAGATGTCC-3′.

Additional experiments suggested that signal may be limited by the amount of probe 2 that has access to the RCA product. Different methods were tested to reduce the diffusion rate of cleaved probe 2. Agarose, glycerol or PEG were included in the cleavage reagent at varying amounts. In one aspect 0.8-2% agarose was added, in another 50-75% glycerol was used with or without the addition of 1M NaCl. The addition of polyethylene glycol (PEG) was also tested, for example 32%. In another aspect a condensation step was added to reduce the diffusion of probe 2. Condensation buffer in the presence of topoisomerase I or MnCl2 was tested. Condensation buffer alone worked better than with the addition of toposiomerase I or MnCl2.

From the foregoing it can be seen that the present invention provides a flexible and scalable method for analyzing complex samples of DNA, such as genomic DNA. These methods are not limited to any particular type of nucleic acid sample: plant, bacterial, animal (including human) total genome DNA, RNA, cDNA and the like may be analyzed using some or all of the methods disclosed in this invention. This invention provides a powerful tool for analysis of complex nucleic acid samples.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

1.-20. (canceled)

21. A method for sequencing a target nucleic acid, the method comprising:
hybridizing the target nucleic acid to a support bound probe, wherein the support bound probe is attached to a solid support, wherein the support bound probe comprises a first probe region and a second probe region, wherein the first probe region comprises a 5′ end and the second probe region comprises a 3′ end, and wherein both the first and second probe regions hybridize to the target nucleic acid;
extending the 3′ end of the second probe region using the hybridized target nucleic acid as a template;
circularizing the support bound probe to create a circularized probe, wherein circularizing comprises ligating the 5′ end of the first probe region with the 3′ end of the second probe region;
digesting support bound probes that have not formed circularized probes;
amplifying the circularized probe to create amplified circularized probes; and
sequencing the amplified circularized probes.

22. The method of claim 21, wherein the amplifying step comprises using rolling circle amplification.

23. The method of 21, wherein the solid support further comprises a plurality of sequencing primers.

24. The method of claim 21, wherein the 3′ end of the second probe region is extended by a single base having a detectable label.

25. The method of claim 21, wherein the first probe region is longer than the second probe region.

26. The method of claim 21, wherein the first probe region is shorter than the second probe region.

27. The method of claim 21, wherein the first probe region comprises a domain that is complementary to a first portion of the target sequence.

28. The method of claim 21, wherein the second probe region comprises a domain that is complementary to a second portion of the target sequence

29. The method of claim 21, wherein the solid support comprises a plurality of beads.

30. The method of claim 21, wherein the solid support comprises a plurality of encoded particles.

31. The method of claim 21, wherein the solid support comprises 1 million to 3 million different support bound probes.

32. The method of claim 21, wherein the solid support comprises a plurality of support probes that interrogate one or more exons.

33. The method of claim 21, wherein the solid support comprises two different probe sequences within each feature.
